(19) United States
(12) Patent Application Publication
(10) Pub. No.: US 2017/0266087 A1
(43) Pub. Date: Sep. 21, 2017

(54) AQUEOUS OR AQUEOUS-ALCOHOLIC GEL OF SYNTHETIC PHYLLOSILICATES AS VISCOSITY-ENHANCING, MATT-EFFECT AND/OR APPLICATION-HOMOGENIZING AGENT

(71) Applicant: L'OREAL, Paris (FR)

(72) Inventors: Bouchra BOUARFA, Chevilly La Rue (FR); Véronique FERRARI, Chevilly La Rue (FR); Fabrice SPRINGINSFELD, Chevilly La Rue (FR); Mathieu CHABRILLANGEAS, Chevilly La Rue (FR); Laurence SEBILLOTTE-ARNAUD, Chevilly La Rue (FR); Raluca LORANT, Chevilly La Rue (FR)

(73) Assignee: L'OREAL, Paris (FR)

(21) Appl. No.: 15/529,384

(22) PCT Filed: Nov. 24, 2015
(86) PCT No.: PCT/EP2015/077519

§ 371 Date: May 24, 2017

(30) Foreign Application Priority Data
Nov. 24, 2014 (FR) 1461334

(51) Int. Cl. A61K 8/25; A61K 8/06; A61Q 19/00; A61K 8/04

(52) U.S. Cl. CPC A61K 8/25; A61K 8/042; A61K 8/062; A61K 8/064; A61Q 19/00; A61K 2800/48

(57) ABSTRACT

The present invention relates to the use of an aqueous or aqueous-alcoholic gel of synthetic phyllosilicate of formula Mg₃Si₄O₁₀(OH)₂ in a cosmetic composition.

A cosmetic composition comprising such a gel and also cosmetic processes for caring for and/or making up the skin and/or the nails using such an aqueous or aqueous-alcoholic gel of synthetic phyllosilicate are also targeted.

The present invention relates to the field of cosmetic products more particularly intended for caring for and/or making up the skin, the nails and the lips and is especially directed toward proposing compositions with increased performance qualities in terms of mattness, viscosity and/or homogeneity after application and advantageously simultaneously satisfying all of these properties.

A large proportion of consumers of cosmetic products intended for caring for or making up the skin, the lips or even the nails now expect products that are capable of affording an effect, in terms of mattness and homogeneity after application, which is improved since it is then liable to be perceived as a natural result. This expectation is especially required in general with regard to care products, but now also for certain products intended for makeup. Moreover, these same consumers maintain their usual requirements as regards these cosmetic products, namely pleasant consistency, easy handling, “play-time” or acceptable spreading qualities in terms of the sensory perception and the duration, which are properties that rather touch upon a rheological aspect.

For obvious reasons, satisfying all these requirements is not immediately achievable by the cosmetic product formulator.

For example, a viscosity that is satisfactory as regards consistency may, on the other hand, be detrimental in terms of the homogeneity result after application. Now, a non-homogeneous result will have an impact on the quality of the deposit. In the process of applying makeup, it may be reflected, for example, by non-homogeneous distribution of the pigments and dyes. Similarly, it may also be detrimental for a care product conveying active agents, for instance UV-screening agents. For obvious reasons, it is important for the care formulation to be uniformly distributed on the skin to facilitate the action of these active agents.

Similarly, it may occur that a performance quality effectively ascertained on normal skin, for example in terms of mattness and homogeneity after application, is not reproduced on greasy skin. Specifically, the substantial secretion of sebum observed on greasy skin has the consequence of generating undesirable skin sheen. On such skin types, a homogeneous and natural result, and thus a generally matt result, afforded by a care or makeup product has an unfortunate tendency to be degraded visually in the course of the day. The initial matt result and similarly the homogeneous effect are thus impaired.

Consequently it is difficult at the present time to provide a care or makeup cosmetic product which, on the one hand, satisfies all these expectations, and, on the other hand, has performance qualities that can be ascertained on a very large number of consumers. Thus, among these consumers are also users whose skin bears wrinkles, fine lines or pores. The homogeneous and natural result expected by these users also usually requires a reduction in the perception of the relief of the treated or made-up surface.

To satisfy these various user expectations, use is conventionally made in cosmetic compositions of mineral or organic fillers of diverse nature. Specifically, depending on their chemical nature and their physicochemical properties, they allow certain consumer expectations to be targeted more particularly. For example, fillers with light-scattering optical properties, known as the soft-focus effect, make it possible to optically smooth out the microrelief and to hide skin imperfections. Other fillers known for their capacity for absorbing sebum and perspiration are favored for providing a matt effect over time.

As representatives of the fillers most commonly used, mention may be made especially of specific fillers such as hydrophobic silica particles (hydrophobic silica aerogels) as described in WO 2013/190112, WO 2013/160362, WO 13190104 or WO 13194100, or silicas functionalized with anionic groups as described in US 2005/0192366. Mention may also be made of certain micas coated with inorganic materials and/or polymethyl methacrylate (PMMA), starch, nylon powders, polyethylene powders, poly-beta-alanine, polymethyl (meth)acrylate powders, boron nitride, or alternatively natural talc with a granulometry (or mean particle size) of 1.8 microns or perlite.

However, none of these fillers can satisfy all of the abovementioned requirements. In addition, the use of some of these fillers may give rise to adverse side effects in certain cosmetic formulations.

Thus, nylon, PMMA, boron nitride, natural talc or silica aerogel powders are difficult to disperse in aqueous medium and may lead to a coarse feel. Perlite has a tendency, after decantation, to form a deposit that it is difficult to re-homogenize when it is introduced into aqueous solutions. As regards natural talc, it has the risk of containing asbestos or heavy metals, which is unfavorable in a cosmetic environment. Moreover, grinding it from a natural block does not make it possible to control its purity and the size of the particles obtained. Furthermore, it is hydrophobic, which does not favor its dispersion in aqueous compositions.

There is thus still a need for a material that can satisfy all of the abovementioned requirements.

In particular, the need remains for a material which, when formulated in a cosmetic product, gives said product improved homogeneity after application without affecting its other performance qualities especially in terms of rheological quality and sensory perception.

The need also remains for a material which has matt-effect and soft-focus performance qualities, and whose effect can be ascertained, over a prolonged period, on a large diversity of skin types, for example normal, greasy, but also wrinkled skin.

There is still also a need to provide a material which has satisfactory properties in terms of dispersion, allowing it to be formulated in a homogeneous and stabilized manner in all the architectures usually considered in the care and makeup sector, while at the same time not significantly impairing the other expected rheological properties.

Contrary to all expectation, the inventors have found that the use of a synthetic phyllosilicate of formula Mg₃Si₄O₁₀(OH)₂ in the form of an aqueous or aqueous-alcoholic gel can specifically satisfy all of these expectations.

Materials of synthetic phyllosilicate type according to the invention are known in the prior art, and are described especially in patent applications FR 2 969 594 and FR 2 925 529. Synthetic phyllosilicates such as those described in patent application WO 2008/009799 and advantageously those disclosed in patent application FR 2 977 580 are most particularly suitable for use in the invention.

However, none of these documents considers exploiting these synthetic phyllosilicates thus obtained in compositions relating to cosmetic, dermatological or pharmaceutical applications.

Thus, according to a first of its aspects, the invention relates to the use of an aqueous or aqueous-alcoholic gel of synthetic phyllosilicate of formula Mg₃Si₄O₁₀(OH)₂ in a cosmetic composition.

According to one embodiment, an aqueous or aqueous-alcoholic gel of synthetic phyllosilicate according to the invention is used as viscosity-enhancing agent.

According to another embodiment, an aqueous or aqueous-alcoholic gel of synthetic phyllosilicate according to the invention is used as matt-effect agent.

According to yet another embodiment, an aqueous or aqueous-alcoholic gel of synthetic phyllosilicate according to the invention is used as homogenizing agent after application, or application homogenizer.

According to another embodiment, an aqueous or aqueous-alcoholic gel according to the invention is used as viscosity-enhancing, matt-effect and application-homogenizing agent.

In addition, the inventors have also observed that an aqueous or aqueous-alcoholic gel according to the invention can give a composition comprising it improved soft-focus properties, i.e. an improved correcting effect on skin imperfections.

Such a gel can also advantageously give the composition comprising it a natural result after application.

Improvement in the wettability of a composition comprising a gel according to the invention has also been observed.

According to another of its aspects, the present invention relates to a composition, especially a cosmetic composition, comprising at least one aqueous or aqueous-alcoholic gel of synthetic phyllosilicate of formula Mg₃Si₄O₁₀(OH)₂ and at least one additional ingredient, different from said synthetic phyllosilicate gel, chosen from silicone fatty substances such as silicone oils, gums and waxes; non-silicone fatty substances such as oils, pasty substances and waxes of plant, mineral, animal and/or synthetic origin; fatty acids containing from 8 to 32 carbon atoms; synthetic esters and ethers; linear or branched hydrocarbons of mineral or synthetic origin; fatty alcohols containing from 8 to 26 carbon atoms; C₂-C₆ alcohols; glycols; surfactants; aqueous or oily gelling agents; cosmetic active agents; fragrances; fillers; dyestuffs; vitamins; preserving agents; film-forming polymers (tensioning or non-tensioning); and mixtures thereof.

Advantageously, a composition according to the invention, comprising said aqueous or aqueous-alcoholic gel of synthetic phyllosilicate, has an infrared absorption band at 7200 cm⁻¹, corresponding to the stretching vibration attributed to the silanol groups Si—OH at the edge of the synthetic phyllosilicate leaflets of the invention.

Advantageously, a composition according to the invention is characterized by an absence of absorption band at 7156 cm¹. This band corresponds to the vibration band of Mg₂FeOH.

A composition according to the present invention also preferably has an infrared absorption band at 7184 cm⁻¹ corresponding to the stretching vibration 2ν Mg₃OH.

It should be noted that in the presence of adsorbed water, for example residual water, an infrared absorption band is detectable and readily identifiable, for example at 5500 cm⁻¹.

According to an implementation variant, the composition according to the invention is a cosmetic or dermatological composition comprising a physiologically acceptable medium.

According to an implementation variant, a composition according to the invention may advantageously comprise, as additional ingredient, at least one compound chosen from lipophilic fillers, pigments, especially hydrophobic pigments, hydrophobic polymers, apolar or silicone oils, and mixtures thereof.

The presence of such an additional ingredient in fact proves to be particularly advantageous when the composition according to the invention is directed toward affording a matt effect.

According to another of its aspects, the invention relates to a cosmetic process for making up and/or caring for the skin and/or the nails, comprising at least one step of applying to said skin and/or said nails a composition comprising, in a physiologically acceptable medium, at least one aqueous or aqueous-alcoholic gel of synthetic phyllosilicate of formula Mg₃Si₄O₁₀(OH)₂. Such a composition may especially be as described later in the present text.

The term “skin” means the skin of the face and/or of the body and semimucous membranes (lips). Preferably, said skin will be the skin of the face and/or of the body and/or of the lips.

For obvious reasons, a composition according to the invention intended to be applied to the nails also means a composition intended to be applied to false nails, insofar as the desired cosmetic effects are often identical.

According to a preferred variant, said process is directed toward affording a matt and/or homogeneous makeup result on the skin complexion.

Preferably, the skin, and better still facial skin, may be exemplified by greasy or shiny skin.

### Synthetic Phyllosilicate According to the Invention

The synthetic phyllosilicate in accordance with the invention has a crystalline structure in accordance with that of a hydroxylated magnesium silicate of molecular formula Mg₃Si₄O₁₀(OH)₂ belonging to the chemical family of phyllosilicates.

These phyllosilicates are generally formed from a stack of elemental leaflets of crystalline structure, the number of which ranges from a few units to several tens of units. Each elemental leaflet is formed by the association of two layers of tetrahedra in which the silicon atoms are positioned, located on either side of a layer of octahedra in which the magnesium atoms are positioned. This group corresponds to the 2/1 phyllosilicates, which are also termed as being of T.O.T. (tetrahedron-octahedron-tetrahedron) type.

As presented above, a synthetic phyllosilicate in accordance with the invention may be obtained according to a preparation process such as the one described in patent application WO 2008/009799 and is preferentially obtained according to the technology described in patent application FR 2 977 580.

This preparation process especially comprises a prolonged hydrothermal treatment, which makes it possible to obtain an aqueous gel of synthetic phyllosilicate. Thus, the synthetic phyllosilicate according to the invention is used in the form of an aqueous or aqueous-alcoholic gel, especially like the one obtained directly on conclusion of the synthetic process.

As described in patent application FR 2 977 580, the parameters that influence the synthesis and the properties of a synthetic phyllosilicate in gel form that is suitable for use in the invention are the nature of the heat treatment (200° C. to 900° C.), the pressure, the nature of the reagents and the proportions thereof.

More particularly, the duration and temperature of the hydrothermal treatment make it possible to control the size of the particles. For example, the lower the temperature, the smaller the synthesized particles, as described in patent application FR 2 977 580. Controlling the size makes it possible to afford new properties and better control of both its hydrophilic and hydrophobic properties, i.e. amphiphilic properties.

Structural Analysis and Characterization of a Synthetic Phyllosilicate that is Suitable for Use in the Invention

A synthetic phyllosilicate that is suitable for use in the invention may be characterized by various parameters, namely infrared absorption bands, its size and its purity, as detailed below.

Under certain conditions, analyses such as nuclear magnetic resonance in particular of 29Si may be useful for the characterization of a synthetic phyllosilicate that is suitable for use in the invention. Similarly, thermogravimetric analysis (TGA) may be used for the characterization of a synthetic phyllosilicate that is suitable for use in the invention. Finally, x-ray diffraction may also be used for this purpose.

Infrared

Method Used The machine used is a Nicolet 6700 FTIR Fourier transform spectrometer, equipped with an integration sphere, with an InGaA detector and a CaF2 separator and a resolution of 12 cm⁻¹, more preferentially of 8 cm⁻¹ and even more preferentially of 4 cm⁻¹. In other words, the values of the absorption bands given in this description should be considered as being plus or minus 6 cm⁻¹ and more preferentially plus or minus 4 cm⁻¹ and even more preferentially plus or minus 2 cm⁻¹.

The near infrared recordings of the stretching region located at 7184 cm⁻¹ were broken down by pseudo-Voigt functions using the Fityk software (Wojdyr, 2010). To visualize the absorption spectrum in a composition comprising at least one aqueous part, such as an emulsion, it is recommended to heat this composition to a temperature corresponding to a temperature of greater than or equal to 100° C. (for example 120° C.) and less than or equal to 500° C. (for example 400° C.) so as to remove the adsorbed water part and, where appropriate, some or all of the organic compound(s) present in the composition.

Generally, to confirm an absorption band, a person skilled in the art performs stretching amplifications. In particular, said person may, for example, make such amplifications of plus or minus 200 cm⁻¹ on either side of a suspected absorption band.

A natural talc is a mineral species composed of doubly hydroxylated magnesium silicate of formula Mg₃Si₄O₁₀(OH)₂, which may contain traces of nickel, iron, aluminum, calcium or sodium.

Natural talc has an infrared spectrum with a typical, fine and strong absorption band at 7184 cm⁻¹ corresponding to the stretching vibration 2ν Mg₃OH. Natural talc generally contains chemical elements which replace magnesium and silicon in the crystalline structure and impose the appearance of at least one additional absorption band, in particular that corresponding to the stretching vibration at 7156 cm⁻¹ attributable to 2ν Mg₂FeOH.

The spectrum of the synthetic phyllosilicate that is suitable for use in the invention differs from that of a natural talc by an absorption band at 7200 cm⁻¹ corresponding to the stretching vibration attributed to the silanol groups Si—OH at the edge of the leaflets.

To confirm this absorption band, a person skilled in the art may perform a stretching amplification, in particular in the region 7400 cm⁻¹-7000 cm⁻¹ and more particularly in the region 7300 cm⁻¹-7100 cm⁻¹.

Preferably, the spectrum of the synthetic phyllosilicate according to the invention is also characterized in that it does not have an absorption band at 7156 cm⁻¹, corresponding to the absorption band of Mg₂FeOH.

Preferably, the spectrum of the synthetic phyllosilicate according to the invention is also characterized by the absorption band at 7184 cm⁻¹ which is common to natural talc.

It should be noted that in the presence of adsorbed water, for example residual water, a broad absorption band is detectable and readily identifiable, for example at 5500 cm⁻¹.

In addition, a composition according to the present invention, comprising said synthetic phyllosilicate has an infrared absorption band at 7200 cm⁻¹, corresponding to the stretching vibration attributed to the silanol groups Si—OH at the edge of the leaflets.

Advantageously, the composition according to the present invention comprising said synthetic phyllosilicate is characterized by the absence of an infrared absorption band at 7156 cm⁻¹, corresponding to the vibration band of Mg₂FeOH.

The composition according to the present invention comprising said synthetic phyllosilicate also preferably has an infrared absorption band at 7184 cm⁻¹, corresponding to the stretching vibration 2ν Mg₃OH.

In a composition according to the invention, it should be noted that in the presence of adsorbed water, for example residual water, a broad infrared absorption band is detectable and readily identifiable, for example at 5500 cm⁻¹.

Size

Method Used

In order to perform the particle size analysis of the synthetic phyllosilicates that are suitable for use in the invention, photon correlation spectroscopy was used. This analytical technique affords access to the size of the particles on the basis of the principle of dynamic light scattering. This device measures over time the intensity of the light scattered by the particles at an angle θ under consideration and the scattered rays are then processed using the Padé-Laplace algorithm.

This non-destructive technique requires dissolution of the particles. The particle size measurement obtained by this technique corresponds to the value of the hydrodynamic diameter of the particle, i.e. it comprises both the size of the particle and also the thickness of the hydration layer.

The analyses were performed using a VASC0-2 particle size analyzer from Cordouan. For the purpose of obtaining statistical information regarding the particle distribution, the NanoQ™ software was used in multi-acquisition mode with the Padé-Laplace algorithm.

Thus, a synthetic phyllosilicate that is suitable for use in the invention, in the form of an aqueous or aqueous-alcoholic gel, advantageously has a mean size ranging from 300 nm to 500 nm.

These characteristics are advantageous with regard to a natural talc, one of the constraints of which is the uncontrolled size of its particles.

Purity

The synthetic phyllosilicate under consideration according to the invention has a degree of purity of at least 99.90% and preferably of at least 99.99%.

It is thus advantageously free of impurities or of undesirable compounds, among which are especially asbestos minerals such as asbestos (serpentine), chlorite, carbonates, heavy metals, iron sulfides, etc., which are generally associated with natural talc and/or incorporated into the structure of natural talcs.

NMR (Nuclear Magnetic Resonance)

Methods Used

The silicon-29 (²⁹Si) NMR spectra were recorded on a Bruker Avance 400 (9.4 T) spectrometer. The reference for the chemical shifts is tetramethylsilane (TMS). The samples were placed in 4 mm zirconia rotors. The magic angle spinning (MAS) speed was set at 8 kHz. The experiments were performed at a room temperature of 21° C.

The ²⁹Si spectra were obtained either by direct polarization (rotation of 30°) with a recycling delay of 60 seconds, or by cross polarization (CP) between 1H and 29Si (recycling time of 5 seconds and contact time of 3 ms).

In silicon (²⁹Si) NMR, natural talc has a single peak at −97 ppm.

In silicon (²⁹Si) NMR, in contrast with natural talc, the spectrum of the synthetic phyllosilicate in accordance with the invention shows two peaks: one located at −95 ppm and the other located at −97 ppm, without the need for particle size fractioning to a size of less than 500 nm.

TGA (Thermogravimetric Analysis)

Method Used

The recordings were made using a Perkin Elmer Diamonds thermobalance.

For each analysis, about 20 mg of sample were required. During the analysis, the sample is subjected to a temperature increase ranging from 30° C. to 1200° C. at a rate of 10° C.·min⁻¹ under a stream of 100 mL·min⁻¹ of air.

The thermogravimetric analysis of a synthetic phyllosilicate in accordance with the invention shows lower thermal stability (at about 800° C.) than that of natural talc and it is characterized by four losses of mass, in contrast with natural talc which has only one, at about 900° C.

To establish these losses of mass, it is useful to refer to the article by Angela Dumas, François Martin, Christophe Le Roux, Pierre Micoud, Sabine Petit, Eric Ferrage, Jocelyne Brendle, Olivier Grauby and Mike Greenhill-Hooper: “Phyllosilicates synthesis: a way of accessing edges contributions in NMR and FTIR spectroscopies. Example of synthetic talc”, Phys. Chem. Minerals, published on 27 Feb. 2013.

X-Ray Diffraction

Method Used

Analysis of the x-ray diffractogram, especially with the aid of the materials and method used for x-ray diffraction analysis, is detailed in patent application FR 2 977 580.

Preferably, given that x-ray diffraction is only performed on solids, to visualize the absorption spectrum in a composition comprising at least one aqueous part, such as an emulsion, it is recommended to heat this composition to a temperature corresponding to a temperature of greater than or equal to 100° C. (for example 120° C.) and less than or equal to 500° C. (for example 400° C.) so as to remove the adsorbed water part and, where appropriate, some or all of the organic compound(s) present in the composition.

The x-ray diffractogram of the synthetic phyllosilicate that is suitable for use in the invention has the same positions of the diffraction lines as those of natural talc, with the exception of one line. Specifically, natural talc has a diffraction line at 9.36 Å whereas the synthetic phyllosilicate in accordance with the invention has a diffraction line above 9.4 Å, which may be up to 9.8 Å.

More particularly, the synthetic phyllosilicate in accordance with the invention has a diffraction line greater than 9.4 Å and less than or equal to 9.8 Å.

The synthetic phyllosilicate in accordance with the invention preferably has a diffraction line greater than or equal to 9.5 Å, advantageously greater than or equal to 9.6 Å, and preferentially greater than or equal to 9.7 Å.

The synthetic phyllosilicate in accordance with the invention preferably has a diffraction line less than or equal to 9.7 Å, advantageously less than or equal to 9.6 Å, and preferentially less than or equal to 9.5 Å.

The synthetic phyllosilicate in accordance with the invention may also have a diffraction line between 4.60 Å and 4.80 Å, and/or a diffraction line between 3.10 Å and 3.20 Å and/or a diffraction line between 1.51 Å and 1.53 Å.

It should be noted that a synthetic phyllosilicate in accordance with the invention is free of interfoliar cations. Specifically, this characteristic is demonstrated by the absence of an x-ray diffraction line located at a distance of between 12.00 Å and 18.00 Å, usually revealing a swelling phase with interfoliar spaces in which are found interfoliar cations and possible water molecules.

It is understood that, since a synthetic phyllosilicate in accordance with the invention is in gel form, the “weight %” means the “weight % of solids” or “weight % of active material”.

The synthetic phyllosilicate in gel form may be present in a composition according to the invention in a content ranging from 0.01% to 40% by weight of active material, in particular ranging from 0.05% to 30% by weight of active material, preferably ranging from 0.1% to 20% by weight of active material and more preferentially from 0.2% to 10% by weight of active material, relative to the total weight of the composition.

Additional Ingredients

As indicated above, a composition according to the invention also comprises at least one ingredient other than the synthetic phyllosilicate required according to the invention.

This additional ingredient may advantageously be chosen from the compounds and additives conventionally considered for the formulation of cosmetic compositions.

It may thus be at least one ingredient conventionally selected for forming a fatty phase or an aqueous phase in a composition.

Thus, according to a first embodiment, a composition according to the invention comprises as additional ingredient at least one constituent compound of a fatty phase. This compound may be chosen in particular from a polar or apolar, volatile or nonvolatile hydrocarbon-based or silicone oil, a wax, a pasty compound, and a mixture thereof.

According to one embodiment, a composition according to the invention comprises as additional ingredient at least one volatile or nonvolatile silicone oil.

According to one embodiment, a composition according to the invention comprises as additional ingredient at least one hydrocarbon-based oil, this oil being chosen especially from volatile C₈-C₁₆ hydrocarbon-based oils, synthetic ethers containing from 10 to 40 carbon atoms, synthetic esters, polyol esters and pentaerythritol esters, fatty alcohols that are liquid at room temperature, bearing a branched and/or unsaturated carbon chain containing from 8 to 26 carbon atoms, C₁₂-C₂₂ higher fatty acids; and mixtures thereof.

According to one implementation variant, a composition according to the invention comprises as additional ingredient according to the invention at least one constituent compound of an aqueous phase, chosen in particular from alcohols, preferably lower monoalcohols containing from 1 to 5 carbon atoms, and polyols preferably chosen from polyols containing from 2 to 32 carbon atoms.

According to one implementation variant, a composition according to the invention comprises as additional ingredient according to the invention at least one fatty alcohol or an ester.

It may also be an ingredient more particularly considered for structuring and/or stabilizing the architecture of a cosmetic composition, for instance surfactants, gelling agents and fillers.

According to one embodiment, a composition according to the invention comprises as additional ingredient according to the invention at least one compound chosen from nonionic, anionic or amphoteric surfactants; fillers; gelling agents; and mixtures thereof.

It may also be chosen from ingredients intended for affording a particular activity such as cosmetic active agents, for instance moisturizers, anti-aging agents and/or vitamins, ingredients whose purpose is to give the composition a specific visual or sensory effect, like dyestuffs such as pigments, nacres and fragrances, or else to ensure an absence of contamination thereof over time, such as preserving agents.

According to one embodiment, a composition according to the invention comprises as additional ingredient according to the invention at least one compound chosen from cosmetic active agents, and especially from moisturizers, anti-aging agents, bleaching agents, antiperspirants; ionic and nonionic surfactants; vitamins; UV-screening agents; dyestuffs, preferably chosen from pigments and nacres; and fragrances, preferably essential oils.

According to one implementation variant, a composition according to the invention comprises as additional ingredient according to the invention at least crosslinked or non-crosslinked hyaluronic acid, or a derivative or salt thereof, in particular a salt of a monovalent cation thereof chosen from sodium and potassium, or of a multivalent cation thereof chosen from calcium, zinc, copper and manganese.

Needless to say, a composition may contain one or more of the ingredients defined above and as detailed below.

Fatty Phase

A composition according to the invention may also comprise a fatty phase.

For the purposes of the invention, a fatty phase includes any liquid fatty substance, generally oils (also known as liquid or oily fatty phase), or solid fatty substance like waxes or pasty compounds (also known as solid fatty phase).

The fatty phase of a composition according to the invention may be constituted solely of a liquid fatty phase or a solid fatty phase, or may comprise a mixture of a liquid fatty phase and of a solid fatty phase.

This fatty phase may advantageously range from 10% to 90% by weight, in particular from 15% to 60% by weight and preferably from 20% to 40% by weight relative to the total weight of the composition.

According to a particular embodiment, a composition in accordance with the invention may contain less than 5% by weight of fatty phase, or even less than 2% by weight of fatty phase relative to the total weight of the composition, or even may be free of fatty phase.

Thus, a composition according to the invention, and in particular its fatty phase, may contain at least one ingredient chosen from silicone fatty substances such as silicone oils, gums and waxes; non-silicone fatty substances such as hydrocarbon-based or fluoro oils, pasty substances and waxes of plant, mineral, animal and/or synthetic origin; fatty acids containing from 8 to 32 carbon atoms; synthetic esters and ethers; linear or branched hydrocarbons, of mineral or synthetic origin, and fatty alcohols containing from 8 to 26 carbon atoms.

Oils

The term “oil” means any fatty substance that is in liquid form at room temperature (25° C.) and at atmospheric pressure (760 mmHg).

An oily phase that is suitable for preparing the cosmetic compositions according to the invention may comprise at least one polar or apolar hydrocarbon-based or silicone oil, or a mixture thereof.

For the purposes of the present invention, the term “silicone oil” means an oil comprising at least one silicon atom, and in particular at least one Si—O group.

The term “fluoro oil” means an oil comprising at least one fluorine atom.

The term “hydrocarbon-based oil” means an oil mainly containing hydrogen and carbon atoms.

The oils may optionally comprise oxygen, nitrogen, sulfur and/or phosphorus atoms, for example in the form of hydroxyl or acid radicals.

The oils may be volatile or nonvolatile.

They may be of plant, mineral or synthetic origin. According to one implementation variant, oils of plant origin are preferred.

For the purposes of the present invention, the term “nonvolatile oil” means an oil with a vapor pressure of less than 0.13 Pa.

For the purposes of the invention, the term “volatile oil” means any oil that is capable of evaporating on contact with the skin in less than one hour, at room temperature and atmospheric pressure. The volatile oil is a volatile cosmetic compound, which is liquid at room temperature, especially having a nonzero vapor pressure, at room temperature and atmospheric pressure, especially having a vapor pressure ranging from 0.13 Pa to 40 000 Pa (10⁻³ to 300 mmHg), in particular ranging from 1.3 Pa to 13 000 Pa (0.01 to 100 mmHg) and more particularly ranging from 1.3 Pa to 1300 Pa (0.01 to 10 mmHg).

Volatile Oils

According to one embodiment, a composition according to the invention may especially comprise at least one hydrocarbon-based or silicone volatile oil.

A hydrocarbon-based volatile oil may be chosen especially from hydrocarbon-based volatile oils containing from 8 to 16 carbon atoms, branched C₈-C₁₆ alkanes, for instance C₈-C₁₆ isoalkanes (also known as isoparaffins), isododecane, isodecane, isohexadecane and, for example, the oils sold under the trade names Isopar or Permethyl, branched C₈-C₁₆ esters, for instance isohexyl neopentanoate, and mixtures thereof.

Preferably, the hydrocarbon-based volatile oil is chosen from hydrocarbon-based volatile oils containing from 8 to 16 carbon atoms, and mixtures thereof, in particular from isododecane, isodecane and isohexadecane.

Mention may also be made of volatile linear alkanes comprising from 8 to 16 carbon atoms, in particular from 10 to 15 carbon atoms and more particularly from 11 to 13 carbon atoms, for instance n-dodecane (C12) and n-tetradecane (C14) sold by Sasol under the respective references Parafol 12-97 and Parafol 14-97, and also mixtures thereof, the undecane-tridecane mixture, mixtures of n-undecane (C11) and of n-tridecane (C13) obtained in examples 1 and 2 of patent application WO 2008/155 059 from the company Cognis, and mixtures thereof.

A silicone volatile oil may be chosen especially from linear silicone volatile oils such as hexamethyldisiloxane, octamethyltrisiloxane, decamethyltetrasiloxane, tetradecamethylhexasiloxane, hexadecamethylheptasiloxane and dodecamethylpentasiloxane.

A silicone volatile oil may be chosen from cyclic silicone volatile oils, for instance hexamethylcyclotrisiloxane, octamethylcyclotetrasiloxane, decamethylcyclopentasiloxane, cyclohexasiloxane and dodecamethylcyclohexasiloxane.

Nonvolatile Oils

A composition according to the invention may also comprise, as additional ingredient, at least one nonvolatile oil chosen from nonvolatile hydrocarbon-based oils and/or silicone oils.

As nonvolatile hydrocarbon-based oils that may suitable for use in the invention, mention may be made especially of:
hydrocarbon-based oils of plant origin,
synthetic ethers containing from 10 to 40 carbon atoms, such as dicaprylyl ether,
synthetic esters, such as the oils of formula R₁COOR₂, in which R₁ represents a linear or branched fatty acid residue comprising from 8 to 40 carbon atoms and R₂ represents a hydrocarbon-based chain, which is in particular branched, containing from 1 to 40 carbon atoms, on condition that R₁+R₂≧10.

These esters may be chosen especially from esters of alcohol and of fatty acid, for instance cetostearyl octanoate, esters of isopropyl alcohol, such as isopropyl myristate, isopropyl palmitate, ethyl palmitate, 2-ethylhexyl palmitate, isopropyl stearate, octyl stearate, hydroxylated esters, for instance isostearyl lactate, octyl hydroxystearate, alcohol or polyalcohol ricinoleates, hexyl laurate, neopentanoic acid esters, for instance isodecyl neopentanoate, isotridecyl neopentanoate, and isononanoic acid esters, for instance isononyl isononanoate and isotridecyl isononanoate,
polyol esters and pentaerythritol esters, such as dipentaerythrityl tetrahydroxystearate/tetraisostearate,
fatty alcohols that are liquid at room temperature, bearing a branched and/or unsaturated carbon chain containing from 8 to 26 carbon atoms, such as 2-octyldodecanol, isostearyl alcohol, oleyl alcohol, of fatty alcohols that may be used in the context of the present invention, mention may be made especially of lauryl alcohol, myristyl alcohol, cetyl alcohol, stearyl alcohol, isostearyl alcohol, palmityl alcohol, oleyl alcohol, cetearyl alcohol or cetylstearyl alcohol (mixture of cetyl alcohol and stearyl alcohol), behenyl alcohol, erucyl alcohol, arachidyl alcohol, 2-hexyldecyl alcohol and isocetyl alcohol, and mixtures thereof,
C₁₂-C₂₂ higher fatty acids, such as oleic acid, linoleic acid, linolenic acid, and mixtures thereof.

For its part, the nonvolatile silicone oil may be chosen from:
non-phenyl silicone oils, for instance caprylyl methicone or polydimethylsiloxanes (PDMS), and
phenyl silicone oils, for instance phenyl trimethicones, phenyl dimethicones, phenyltrimethyl siloxydiphenyl siloxanes, diphenyl dimethicones, diphenylmethyldiphenyltrisiloxanes and 2-phenylethyl trimethylsiloxysilicates, dimethicones or phenyl trimethicone with a viscosity of less than or equal to 100 cSt, and trimethyl-pentaphenyl-trisiloxane, and mixtures thereof; and also mixtures of these various oils.

Thus, according to one embodiment, a composition according to the invention comprises at least one additional ingredient, said additional ingredient being:
at least one nonvolatile hydrocarbon-based oil chosen from synthetic ethers containing from 10 to 40 carbon atoms, synthetic esters, polyol esters and pentaerythritol esters, fatty alcohols that are liquid at room temperature, bearing a branched and/or unsaturated carbon chain containing from 8 to 26 carbon atoms, C₁₂-C₂₂ higher fatty acids; and/or
at least one nonvolatile silicone oil chosen from non-phenyl silicone oils and phenyl silicone oils, and/or
a mixture thereof.

Preferably, a composition according to the invention comprises as additional ingredient according to the invention at least one volatile and/or nonvolatile silicone oil.

A composition according to the invention may also comprise one or more additional ingredients dissolved in oils such as silicone resins, for instance trifluoromethyl-C₁₋₄-alkyl dimethicone and trifluoropropyl dimethicone, and silicone elastomers, for instance the products sold under the KSG names by the company Shin-Etsu, under the name Trefil by the company Dow Corning or under the Gransil names by the company Grant Industries; and mixtures thereof.

The oily phase according to the invention may in particular have a threshold stress of greater than 1.5 Pa and preferably greater than 10 Pa. This threshold stress value reflects a gel-type texture of this oily phase.

A composition according to the invention may also comprise at least one solid fatty substance chosen from hydrocarbon-based or silicone gums and waxes, pasty substances and waxes of plant, mineral and/or synthetic origin.

Waxes

For the purposes of the present invention, the term “wax” means a lipophilic fatty compound that is solid at room temperature (25° C.), with a reversible solid/liquid change of state, having a melting point of greater than 30° C. which may be up to 200° C., a hardness of greater than 0.5 MPa, and having an anisotropic crystal organization in the solid state. By bringing the wax to its melting point, it is possible to make it miscible with oils and to form a microscopically homogeneous mixture, but on returning the temperature of the mixture to room temperature, recrystallization of the wax in the oils of the mixture is obtained.

The waxes that may be used in the invention are compounds that are solid at room temperature, which are intended to structure the composition, in particular in stick form; they may be hydrocarbon-based, fluoro-based and/or silicone-based and may be of plant, mineral and/or synthetic origin. In particular, they have a melting point of greater than 40° C. and better still greater than 45° C.

As wax that may be used in the invention, mention may be made of those generally used in cosmetics: they are especially of natural origin, such as beeswax, carnauba wax, candelilla wax, ouricury wax, Japan wax, cork fiber wax or sugarcane wax, rice wax, montan wax, paraffin, lignite wax or microcrystalline wax, ceresin or ozokerite, hydrogenated oils such as jojoba oil; synthetic waxes such as polyethylene waxes derived from the polymerization or copolymerization of ethylene and Fischer-Tropsch waxes, or alternatively fatty acid esters such as octacosanyl stearate, glycerides that are solid at 40° C. and better still at 45° C., silicone waxes such as alkyl or alkoxy dimethicones with an alkyl or alkoxy chain of 10 to 45 carbon atoms, poly(di)methylsiloxane esters that are solid at 40° C., the ester chain of which comprises at least 10 carbon atoms; and mixtures thereof.

Pasty Compound

For the purposes of the present invention, the term “pasty” is intended to denote a lipophilic fatty compound with a reversible solid/liquid change of state, and comprising at a temperature of 25° C. a liquid fraction and a solid fraction.

A pasty compound is advantageously chosen from:
lanolin and derivatives thereof,
polymeric or non-polymeric fluoro compounds,
polymeric or non-polymeric silicone compounds,
vinyl polymers, especially:
olefin homopolymers,
olefin copolymers,
hydrogenated diene homopolymers and copolymers,
linear or branched homopolymer or copolymer oligomers of alkyl (meth)acrylates preferably bearing a C₈-C₃₀ alkyl group,
homopolymer and copolymer oligomers of vinyl esters bearing C₈-C₃₀ alkyl groups,
homopolymer and copolymer oligomers of vinyl ethers bearing C₈-C₃₀ alkyl groups,
liposoluble polyethers resulting from polyetherification between one or more C₂-C₁₀₀ and preferably C₂-C₅₀ diols,
esters,
polyvinyl laurate; and
mixtures thereof.

Aqueous Phase

As emerges from the foregoing, a composition according to the invention contains an aqueous phase formed at least partly by the aqueous or aqueous-alcoholic gel of synthetic phyllosilicate.

According to one embodiment, the aqueous or aqueous-alcoholic gel of synthetic phyllosilicate according to the invention forms the aqueous phase, i.e. the aqueous phase is constituted exclusively of this gel.

Advantageously, a composition according to the invention also contains at least one additional hydrophilic ingredient.

Thus, the aqueous phase of a composition according to the invention may also comprise water and/or a water-soluble solvent.

In the present invention, the term “water-soluble solvent” denotes a compound that is liquid at room temperature and water-miscible (miscibility with water of greater than 50% by weight at 25° C. and atmospheric pressure).

The water-soluble solvents that may be used in the composition of the invention may also be volatile.

Thus, a composition according to the invention may comprise at least one water-soluble solvent chosen from lower monoalcohols containing from 1 to 5 carbon atoms such as ethanol and isopropanol and polyols.

The polyols that are advantageously suitable for formulating a composition according to the present invention are those especially containing from 2 to 32 carbon atoms and preferably 3 to 16 carbon atoms.

Advantageously, the polyol may be chosen, for example, from ethylene glycol, pentaerythritol, trimethylolpropane, propylene glycol, 1,3-propanediol, butylene glycol, isoprene glycol, pentylene glycol, hexylene glycol, glycerol, polyglycerols, such as glycerol oligomers, for instance diglycerol, and polyethylene glycols, and mixtures thereof.

According to a preferred embodiment of the invention, said polyol is chosen from ethylene glycol, pentaerythritol, trimethylolpropane, propylene glycol, butylene glycol, glycerol, polyglycerols and polyethylene glycols, and mixtures thereof.

According to a particular mode, the composition of the invention may comprise at least butylene glycol and/or glycerol.

The aqueous phase may be present in the composition in a content ranging from 5% to 98%, 5% to 95%, better still from 30% to 80% by weight and preferably from 40% to 75% by weight relative to the total weight of said composition.

Ingredients Chosen from Surfactants, Gelling Agents and Fillers

Surfactants

A composition according to the invention may comprise from 0.1% to 10% by weight, preferably from 0.5% to 5% by weight and in particular from 1.0% to 4% by weight of surfactant(s) relative to the total weight of the composition.

The surfactants may be chosen from nonionic, anionic and amphoteric surfactants, and mixtures thereof. Reference may be made to Kirk-Othmer's Encyclopedia of Chemical Technology, volume 22, pages 333-432, 3rd edition, 1979, Wiley, for the definition of the emulsifying properties and functions of surfactants, in particular pages 347-377 of this reference, for the anionic, amphoteric and nonionic surfactants.

A composition according to the invention may comprise at least one hydrocarbon-based surfactant, a silicone surfactant, and a mixture thereof.

Examples of hydrocarbon-based surfactants that are suitable for use in the invention are described below.

Nonionic Surfactants

The nonionic surfactants may be chosen especially from alkyl and polyalkyl esters of poly(ethylene oxide), oxyalkylenated alcohols, alkyl and polyalkyl ethers of poly(ethylene oxide), optionally polyoxyethylenated alkyl and polyalkyl esters of sorbitan, optionally polyoxyethylenated alkyl and polyalkyl ethers of sorbitan, alkyl and polyalkyl glycosides or polyglycosides, in particular alkyl and polyalkyl glucosides or polyglucosides, alkyl and polyalkyl esters of sucrose, optionally polyoxyethylenated alkyl and polyalkyl esters of glycerol, and optionally polyoxyethylenated alkyl and polyalkyl ethers of glycerol, and mixtures thereof.

1) Alkyl and polyalkyl esters of poly(ethylene oxide) that are preferably used are those with a number of ethylene oxide (EO) units ranging from 2 to 200. Examples that may be mentioned include stearate 40 EO, stearate 50 EO, stearate 100 EO, laurate 20 EO, laurate 40 EO and distearate 150 EO.

2) Alkyl and polyalkyl ethers of poly(ethylene oxide) that are preferably used are those with a number of ethylene oxide (EO) units ranging from 2 to 200. Examples that may be mentioned include cetyl ether 23 EO, oleyl ether 50 EO, phytosterol 30 EO, steareth 40, steareth 100 and beheneth 100.

3) Oxyalkylenated alcohols, which are in particular oxyethylenated and/or oxypropylenated, that are preferably used are those that can comprise from 1 to 150 oxyethylene and/or oxypropylene units, in particular containing from 20 to 100 oxyethylene units, in particular ethoxylated C₈-C₂₄ and preferably C₁₂-C₁₈ fatty alcohols, such as stearyl alcohol ethoxylated with 20 oxyethylene units (CTFA name Steareth-20), for instance Brij 78 sold by the company Uniqema, cetearyl alcohol ethoxylated with 30 oxyethylene units (CTFA name Ceteareth-30), and the mixture of C₁₂-C₁₅ fatty alcohols comprising 7 oxyethylene units (CTFA name C₁₂-C₁₅ Pareth-7), for instance the product sold under the name Neodol 25-7® by Shell Chemicals; or in particular oxyalkylenated (oxyethylenated and/or oxypropylenated) alcohols containing from 1 to 15 oxyethylene and/or oxypropylene units, in particular ethoxylated C₈-C₂₄ and preferably C₁₂-C₁₈ fatty alcohols, such as stearyl alcohol ethoxylated with 2 oxyethylene units (CTFA name Steareth-2), for instance Brij 72 sold by the company Uniqema;

4) Optionally polyoxyethylenated alkyl and polyalkyl esters of sorbitan that are preferably used are those with a number of ethylene oxide (EO) units ranging from 0 to 100. Examples that may be mentioned include sorbitan laurate 4 or 20 EO, in particular polysorbate 20 (or polyoxyethylene (20) sorbitan monolaurate) such as the product Tween 20 sold by the company Uniqema, sorbitan palmitate 20 EO, sorbitan stearate 20 EO, sorbitan oleate 20 EO, or the Cremophor products (RH 40, RH 60, etc.) from BASF.

5) Optionally polyoxyethylenated alkyl and polyalkyl ethers of sorbitan that are preferably used are those with a number of ethylene oxide (EO) units ranging from 0 to 100.

6) Alkyl and polyalkyl glucosides or polyglucosides that are preferably used are those containing an alkyl group comprising from 6 to 30 carbon atoms and preferably from 6 to 18 or even from 8 to 16 carbon atoms, and containing a glucoside group preferably comprising from 1 to 5 and in particular 1, 2 or 3 glucoside units. The alkylpolyglucosides may be chosen, for example, from decyl glucoside (alkyl-C₉/Cᵢᵢ-polyglucoside (1.4)), for instance the product sold under the name Mydol 10® by the company Kao Chemicals or the product sold under the name Plantacare 2000 UP® by the company Henkel and the product sold under the name Oramix NS 10® by the company SEPPIC; caprylyl/capryl glucoside, for instance the product sold under the name Plantacare KE 3711® by the company Cognis or Oramix CG 110® by the company SEPPIC; laurylglucoside, for instance the product sold under the name Plantacare 1200 UP® by the company Henkel or Plantaren 1200 N® by the company Henkel; cocoglucoside, for instance the product sold under the name Plantacare 818 UP® by the company Henkel; caprylylglucoside, for instance the product sold under the name Plantacare 810 UP® by the company Cognis; or surfactants such as the alkylpolyglucosides sold under the commercial reference Montanov; and mixtures thereof.

More generally, the surfactants of alkylpolyglycoside type are defined more specifically hereinbelow.

7) Examples of alkyl and polyalkyl esters of sucrose that may be mentioned are Crodesta F150, sucrose monolaurate sold under the name Crodesta SL 40, and the products sold by Ryoto Sugar Ester, for instance sucrose palmitate sold under the reference Ryoto Sugar Ester P1670, Ryoto Sugar Ester LWA 1695 or Ryoto Sugar Ester 01570.

8) Optionally polyoxyethylenated alkyl and polyalkyl esters of glycerol that are preferably used are those with a number of ethylene oxide (EO) units ranging from 0 to 100 and a number of glycerol units ranging from 1 to 30. Examples that may be mentioned include hexaglyceryl monolaurate and PEG-30 glyceryl stearate.

9) Optionally polyoxyethylenated alkyl and polyalkyl ethers of glycerol that are preferably used are those with a number of ethylene oxide (EO) units ranging from 0 to 100 and a number of glycerol units ranging from 1 to 30. Examples that may be mentioned include Nikkol Batyl Alcohol 100 and Nikkol Chimyl Alcohol 100.

A nonionic surfactant according to the invention that may also be mentioned is a glyceryl mono/distearate/polyethylene glycol (100 EO) stearate mixture.

Anionic Surfactants

The anionic surfactants may be chosen from alkyl ether sulfates, carboxylates, amino acid derivatives, sulfonates, isethionates, taurates, sulfosuccinates, alkylsulfoacetates, phosphates and alkyl phosphates, polypeptides, metal salts of C₁₀-C₃₀ and in particular C₁₂-C₂₀ fatty acids, in particular metal stearates, and mixtures thereof.

1) Examples of alkyl ether sulfates that may be mentioned include sodium lauryl ether sulfate (70/30 C12-C14) (2.2 EO) sold under the names Sipon AOS225 or Texapon N702 by the company Henkel, ammonium lauryl ether sulfate (70/30 C12-C14) (3 EO) sold under the name Sipon LEA 370 by the company Henkel, ammonium (C₁₂-C₁₄) alkyl ether (9 EO) sulfate sold under the name Rhodapex AB/20 by the company Rhodia Chimie, and the mixture of sodium and magnesium lauryl and oleyl ether sulfate sold under the name Empicol BSD 52 by the company Albright & Wilson.

2) Examples of carboxylates that may be mentioned include salts (for example alkali salts) of N-acylamino acids, glycol carboxylates, amido ether carboxylates (AEC) and polyoxyethylenated carboxylic acid salts.

The surfactant of glycol carboxylate type may be chosen from alkyl glycol carboxylics or 2-(2-hydroxyalkyloxyacetate), salts thereof and mixtures thereof. These alkyl glycol carboxylics comprise a linear or branched, saturated or unsaturated, aliphatic and/or aromatic alkyl chain containing from 8 to 18 carbon atoms. These carboxylics may be neutralized with mineral bases such as potassium hydroxide or sodium hydroxide.

Examples of surfactants of glycol carboxylic type that may be mentioned include sodium lauryl glycol carboxylate or sodium 2-(2-hydroxyalkyloxy acetate) such as the product sold under the name Beaulight Shaa® by the company Sanyo, Beaulight LCA-25N® or the corresponding acid form Beaulight Shaa (Acid form)®.

An example of an amido ether carboxylate (AEC) that may be mentioned is sodium lauryl amido ether carboxylate (3 EO) sold under the name Akypo Foam 30® by the company Kao Chemicals.

Examples of polyoxyethylenated carboxylic acid salts that may be mentioned include oxyethylenated (6 EO) sodium lauryl ether carboxylate (65/25/10 C₁₂₋₁₄₋₁₆) sold under the name Akypo Soft 45 NV® by the company Kao Chemicals, polyoxyethylenated and carboxymethylated fatty acids of olive oil origin sold under the name Olivem 400® by the company Biologia e Tecnologia, and oxyethylenated (6 EO) sodium tridecyl ether carboxylate sold under the name Nikkol ECTD-6 NEX® by the company Nikkol.

3) Amino acid derivatives that may especially be mentioned include alkaline salts of amino acids, such as:
sarcosinates, for instance the sodium lauroyl sarcosinate sold under the name Sarkosyl NL 97® by the company Ciba or sold under the name Oramix L30® by the company SEPPIC, sodium myristoyl sarcosinate sold under the name Nikkol Sarcosinate MN® by the company Nikkol, and sodium palmitoyl sarcosinate sold under the name Nikkol Sarcosinate PN® by the company Nikkol;
alaninates, for instance sodium N-lauroyl N-methyl amidopropionate sold under the name Sodium Nikkol Alaninate LN 30® by the company Nikkol, or sold under the name Alanone ALE® by the company Kawaken, and triethanolamine N-lauroyl N-methyl alanine sold under the name Alanone Alta® by the company Kawaken;
glutamates, for instance triethanolamine monococoyl glutamate sold under the name Acylglutamate CT-12® by the company Ajinomoto, or triethanolamine lauroyl glutamate sold under the name Acylglutamate LT-12® by the company Ajinomoto.

The glutamic acid salts and/or derivatives are described more specifically hereinbelow;
aspartates, for instance the mixture of triethanolamine N-lauroyl aspartate and of triethanolamine N-myristoyl aspartate, sold under the name Asparack® by the company Mitsubishi;
glycine derivatives (glycinates), for instance the sodium N-cocoyl glycinate sold under the names Amilite GCS-12® and Amilite GCK 12 by the company Ajinomoto;
citrates, such as the citric monoester of oxyethylenated (9 mol) coco alcohols sold under the name Witconol EC 1129 by the company Goldschmidt;
galacturonates, such as the sodium dodecyl-D-galactoside uronate sold by the company Soliance.

4) Examples of sulfonates that may be mentioned include α-olefin sulfonates, for instance the sodium α-olefin sulfonate (C₁₄₋₁₆) sold under the name Bio-Terge AS 40® by the company Stepan, sold under the names Witconate AOS Protégé® and Sulframine AOS PH 12® by the company Witco or sold under the name Bio-Terge AS 40 CG® by the company Stepan, the sodium secondary olefin sulfonate sold under the name Hostapur SAS 30® by the company Clariant.

5) Isethionates that may be mentioned include acylisethionates, for instance sodium cocoylisethionate, such as the product sold under the name Jordapon CI P® by the company Jordan.

6) Taurates that may be mentioned include the sodium salt of palm kernel oil methyltaurate sold under the name Hostapon CT Pate® by the company Clariant; N-acyl N-methyltaurates, for instance the sodium N-cocoyl N-methyltaurate sold under the name Hostapon LT-SF® by the company Clariant or sold under the name Nikkol CMT-30-T® by the company Nikkol, and the sodium palmitoyl methyltaurate sold under the name Nikkol PMT® by the company Nikkol.

7) Examples of sulfosuccinates that may be mentioned include the oxyethylenated (3 EO) lauryl alcohol monosulfosuccinate (70/30 C₁₂/C₁₄) sold under the names Setacin 103 Special® and Rewopol SB-FA 30 K 4® by the company Witco, the disodium salt of a C₁₂-C₁₄ alcohol hemisulfosuccinate, sold under the name Setacin F Special Paste® by the company Zschimmer Schwarz, the oxyethylenated (2 EO) disodium oleamidosulfosuccinate sold under the name Standapol SH 135® by the company Henkel, the oxyethylenated (5 EO) laurylamide monosulfosuccinate sold under the name Lebon A-5000® by the company Sanyo, the oxyethylenated (10 EO) disodium salt of lauryl citrate monosulfosuccinate sold under the name Rewopol SB CS 50® by the company Witco, and the ricinoleic monoethanolamide monosulfosuccinate sold under the name Rewoderm S 1333® by the company Witco. Polydimethylsiloxane sulfosuccinates may also be used, such as the disodium PEG-12 dimethicone sulfosuccinate sold under the name Mackanate-DC30 by the company Maclntyre.

8) Examples of alkyl sulfoacetates that may be mentioned include the mixture of sodium lauryl sulfoacetate and disodium lauryl ether sulfosuccinate, sold under the name Stepan-Mild LSB by the company Stepan.

9) Examples of phosphates and alkyl phosphates that may be mentioned include monoalkyl phosphates and dialkyl phosphates, such as the lauryl monophosphate sold under the name MAP 20® by the company Kao Chemicals, the potassium salt of dodecylphosphoric acid, mixture of monoester and diester (predominantly diester) sold under the name Crafol AP-31® by the company Cognis, the mixture of octylphosphoric acid monoester and diester sold under the name Crafol AP-20® by the company Cognis, the mixture of ethoxylated (7 mol of EO) phosphoric acid monoester and diester of 2-butyloctanol, sold under the name Isofol 12 7 EO-Phosphate Ester® by the company Condea, the potassium or triethanolamine salt of mono(C₁₂-C₁₃)alkyl phosphate sold under the references Arlatone MAP 230K-40® and Arlatone MAP 230T-60® by the company Uniqema, the potassium lauryl phosphate sold under the name Dermalcare MAP XC-99/09® by the company Rhodia Chimie, and the potassium cetyl phosphate sold under the name Arlatone MAP 160K by the company Uniqema.

10) The polypeptides are obtained, for example, by condensation of a fatty chain onto amino acids from cereals and in particular from wheat and oat. Examples of polypeptides that may be mentioned include the potassium salt of hydrolyzed lauroyl wheat protein, sold under the name Aminofoam W OR by the company Croda, the triethanolamine salt of hydrolyzed cocoyl soybean protein, sold under the name May-Tein SY by the company Maybrook, the sodium salt of lauroyl oat amino acids, sold under the name Proteol Oat by the company SEPPIC, collagen hydrolyzate grafted onto coconut fatty acid, sold under the name Geliderm 3000 by the company Deutsche Gelatine, and soybean proteins acylated with hydrogenated coconut acids, sold under the name Proteol VS 22 by the company SEPPIC.

11) As metal salts of C₁₀-C₃₀ and especially C₁₂-C₂₀ fatty acids, mention may be made in particular of metal stearates, such as sodium stearate and potassium stearate, and also polyhydroxystearates.

Fillers

The compositions in accordance with the invention may also comprise at least one filler other than the synthetic phyllosilicate. Needless to say, its choice in terms of chemical nature and amount will be adjusted so that the properties imparted by the synthetic phyllosilicate required in parallel according to the invention are not affected.

The fillers may be chosen from those that are well known to persons skilled in the art and commonly used in cosmetic compositions. The fillers may be mineral or organic, and lamellar or spherical.

Mention may be made of talc, mica, hydrophilic or hydrophobic silica, aerogels, perlite, kaolin, polyamide powders, for instance the Nylon® sold under the name Orgasol® by the company Atochem, poly-β-alanine powders and polyethylene powders, powders of tetra-fluoro-ethylene polymers, for instance Teflon®, lauroyllysine, starch, boron nitride, expanded polymeric hollow microspheres such as those of polyvinylidene chloride/acrylonitrile, for instance the products sold under the name Expancel® by the company Nobel Industrie, acrylic powders such as those sold under the name Polytrap® by the company Dow Corning, polymethyl methacrylate particles and silicone resin microbeads (for example Tospearls from Toshiba), precipitated calcium carbonate, magnesium carbonate and magnesium hydrocarbonate, hydroxyapatite, hollow silica microspheres (Silica Beads® from Maprecos), glass or ceramic microcapsules, metal soaps derived from organic carboxylic acids having from 8 to 22 carbon atoms and in particular from 12 to 18 carbon atoms, for example zinc, magnesium or lithium stearate, zinc laurate and magnesium myristate.

The fillers other than the synthetic phyllosilicate according to the invention may represent from 0.001% to 15% by weight and in particular from 0.5% to 10% by weight relative to the total weight of the composition.

Gelling Agents

A gelling agent according to the present invention may be chosen from at least one hydrophilic gelling agent, a lipophilic gelling agent, and mixtures thereof.

The hydrophilic gelling agent may be chosen from montmorillonites, hectorites, bentonites, beidellite, saponites, laponites, fumed silicas, synthetic polymeric gelling agents, polymeric gelling agents that are natural or of natural origin, for instance non-starchy polysaccharides, native starches, modified starches, starches grafted with an acrylic polymer, hydrolyzed starches grafted with an acrylic polymer, polymers based on starch, gum and cellulose derivative, and mixtures thereof.

More particularly, a hydrophilic gelling polymer may be chosen from:
acrylic or methacrylic acid homopolymers or copolymers or salts thereof and esters thereof and in particular the products sold under the names Versicol F or Versicol K by the company Allied Colloid, Ultrahold 8 by the company Ciba-Geigy, and polyacrylic acids of Synthalen K type, and salts, in particular sodium salts, of polyacrylic acids (corresponding to the INCI name Sodium Acrylate Copolymer) and more particularly a crosslinked sodium polyacrylate (corresponding to the INCI name Sodium Acrylate Copolymer (and) Caprylic/Capric Triglyceride) sold under the name Luvigel EM by the company,
copolymers of acrylic acid and of acrylamide sold in the form of the sodium salt thereof under the Reten names by the company Hercules, the sodium polymethacrylate sold under the name Darvan No. 7 by the company Vanderbilt, and the sodium salts of polyhydroxycarboxylic acids sold under the name Hydagen F by the company Henkel,
polyacrylic acid/alkyl acrylate copolymers, preferably modified or unmodified carboxyvinyl polymers; the copolymers most particularly preferred according to the present invention are acrylate/C₁₀-C₃₀-alkylacrylate copolymers (INCI name: Acrylates/C₁₀₋₃₀ Alkyl Acrylate Crosspolymer) such as the products sold by the company Lubrizol under the trade names Pemulen TR1, Pemulen TR2, Carbopol 1382 and Carbopol EDT 2020, and even more preferentially Pemulen TR-2,
AMPS (polyacrylamidomethylpropanesulfonic acid partially neutralized with aqueous ammonia and highly crosslinked) sold by the company Clariant,
AMPS/acrylamide copolymers of Sepigel or Simulgel type sold by the company SEPPIC,
polyoxyethylenated AMPS/alkyl methacrylate copolymers (crosslinked or non-crosslinked) of Aristoflex HMS type sold by the company Clariant,
anionic, cationic, amphoteric or nonionic chitin or chitosan polymers,
cellulose polymers, other than alkylcellulose, chosen from hydroxyethylcellulose, hydroxypropylcellulose, hydroxymethylcellulose, ethylhydroxyethylcellulose and carboxymethylcellulose, and also quaternized cellulose derivatives,
vinyl polymers, for instance polyvinylpyrrolidones, copolymers of methyl vinyl ether and of malic anhydride, the copolymer of vinyl acetate and of crotonic acid, copolymers of vinylpyrrolidone and of vinyl acetate; copolymers of vinylpyrrolidone and of caprolactam;

polyvinyl alcohol,
anionic, cationic, nonionic or amphoteric associative polymers, for instance maleic anhydride/C₃₀-C₃₈ α-olefin/alkyl maleate terpolymers, the methacrylic acid/ethyl acrylate/oxyalkylenated (20 EO units) stearyl methacrylate terpolymer or the methacrylic acid/ethyl acrylate/oxyethylene (25 EO) behenyl methacrylate terpolymer, celluloses modified with groups comprising at least one fatty chain or with polyalkylene glycol alkylphenyl ether groups, copolymers of vinylpyrrolidone and of fatty-chain hydrophobic monomers; copolymers of C₁-C₆ alkyl methacrylates or acrylates and of amphiphilic monomers comprising at least one fatty chain, copolymers of hydrophilic methacrylates or acrylates and of hydrophobic monomers comprising at least one fatty chain, associative polyurethanes, acrylic acid/acrylamidopropyltrimethylammonium chloride/stearyl methacrylate copolymers,
optionally modified polymers of natural origin, such as:

galactomannans and derivatives thereof, such as konjac gum, gellan gum, locust bean gum, fenugreek gum, karaya gum, gum tragacanth, gum arabic, acacia gum, guar gum, hydroxypropyl guar, hydroxypropyl guar modified with sodium methylcarboxylate groups (Jaguar XC97-1, Rhodia), hydroxypropyltrimethylammonium guar chloride, and xanthan derivatives, such as xanthan gum,
alginates and carrageenans,
glycoaminoglycans,
deoxyribonucleic acid,
mucopolysaccharides such as hyaluronic acid and derivatives thereof, in particular sodium hyaluronate, chondroitin sulfates, and mixtures thereof.

According to one embodiment, a hydrophilic gelling agent is chosen from fumed silicas, synthetic polymeric gelling agents, polymeric gelling agents that are natural or of natural origin, acrylic or methacrylic acid homopolymers or copolymers, salts thereof or esters thereof, polyacrylic acid/alkyl acrylate copolymers, crosslinked or non-crosslinked polyoxyethylenated AMPS/alkyl methacrylate copolymers, anionic, cationic, amphoteric or nonionic chitin or chitosan polymers, cellulose polymers, other than alkylcellulose, chosen from hydroxyethylcellulose, hydroxypropyl cellulose, hydroxymethylcellulose, ethylhydroxyethylcellulose, carboxymethylcellulose, and also quaternized cellulose derivatives, vinyl polymers and anionic, cationic, nonionic or amphoteric associative polymers, modified or unmodified polymers of natural origin, alginates and carrageenans, glycoaminoglycans, deoxyribonucleic acid, mucopolysaccharides, in particular hyaluronic acid and derivatives thereof, and chondroitin sulfates, and mixtures thereof.

According to one embodiment, a synthetic phyllosilicate according to the invention used in combination with at least one hyaluronic acid, or a derivative thereof, especially a salt thereof.

This embodiment is especially illustrated in example 6 of the present patent application.

Hyaluronic acid is a linear, non-sulfated glycosaminoglycan composed of D-glucuronic acid and N-acetyl-D-glucosamine repeating units.

According to the invention, the hyaluronic acid or a derivative thereof preferably has a number-average molecular weight ranging from 500 Da to 10 MDa and more particularly ranging from 2 kDa to 2 MDa.

As hyaluronic acid that is suitable for use in the present invention, mention may be made especially of hyaluronic acids of animal origin, or obtained via biotechnology. They are linear or crosslinked, such as those sold under the name Hylaform® by the company Genzyme, or hyaluronic acids of genetic origin, including those intended for periorbital or peri-buccal surface wrinkles, such as those sold under the name Restylane Fine Lines® by Laboratoire Q-Med, or intended for deep wrinkles and labio-mandibular and oval depressions of the face, such as those sold under the names Perlane® and Restylane Sub-Q® by Laboratoire Q-Med.

Preferably, as hyaluronic acid that is suitable for use in the present invention, mention may be made of those sold under the name Restylane® by Laboratoire Q-Med and under the name Surgiderm® by Laboratoire Corneal.

The following forms of sodium hyaluronate may also be advantageously considered in the context of the invention:
of molecular weight between 20 and 50 kg/mol sold by the company Soliance under the name Renovyal LO®,
of molecular weight between 100 and 300 kg/mol sold by the company Soliance under the name Primalhyal 300®,
of molecular weight between 500 and 1200 kg/mol sold by the company Soliance under the name Cristalhyal LO®,
of molecular weight between 10 and 200 kg/mol sold by the company Bioland under the name Bioha®,
of molecular weight between 30 and 60 kg/mol sold by the company Evonik Goldschmidt under the name Hyacare 50®,
of molecular weight between 100 and 140 kg/mol sold by the company Contripro under the name Hyactive 120®.

Among the hyaluronic acid salts, mention may be made especially of the sodium salts, the potassium salts, the zinc salts and the silver salts, and mixtures thereof.

More particularly, as hyaluronic acid salts, mention may be made of potassium hyaluronate and sodium hyaluronate, preferably sodium hyaluronate.

According to another particular embodiment, a composition may comprise at least one lipophilic gelling agent.

A lipophilic gelling agent may be chosen from particulate gelling agents, and especially modified clays, silicas such as fumed silicas and hydrophobic silica aerogels; organopolysiloxane elastomers; semicrystalline polymers; hydrocarbon-based block copolymers; dextrin esters; polymers bearing hydrogen bonding; and mixtures thereof.

According to one embodiment of the invention, a lipophilic gelling agent is chosen from particulate gelling agents, organopolysiloxane elastomers, semicrystalline polymers, hydrocarbon-based block copolymers, dextrin esters and polymers bearing hydrogen bonding, and mixtures thereof.

As representative lipophilic particulate gelling agents that are suitable for use in the invention, mention may be made most particularly of modified clays, and silicas such as fumed silicas and hydrophobic silica aerogels.

a) The clays may be natural or synthetic, and they are made lipophilic by treatment with an alkylammonium salt such as a C₁₀ to C₂₂ ammonium chloride, for example distearyldimethylammonium chloride.

They may be chosen from bentonites, in particular hectorites and montmorillonites, beidellites, saponites, nontronites, sepiolites, biotites, attapulgites, vermiculites and zeolites.

They are preferably chosen from hectorites.

Hectorites modified with a C₁₀ to C₂₂ ammonium chloride, such as hectorite modified with distearyldimethylammonium chloride, for instance the product sold under the name Bentone 38V® by the company Elementis or bentone gel in isododecane sold under the name Bentone Gel ISD V® (87% isododecane/10% disteardimonium hectorite/3% propylene carbonate) by the company Elementis, are preferably used as lipophilic clays.

The lipophilic clay may especially be present in a content ranging from 0.1% to 15% by weight, in particular from 0.50.1% to 10% and more particularly from 0.2% to 8% by weight relative to the total weight of the composition.

b) The fumed silica is advantageously hydrophobically surface-treated. The hydrophobic groups may be:
trimethylsiloxyl groups, which are obtained in particular by treatment of fumed silica in the presence of hexamethyldisilazane. Silicas thus treated are known as “Silica Silylate” according to the CTFA (8th edition, 2000). They are sold, for example, under the references Aerosil R812® by the company Degussa, and Cab-O-Sil TS-530® by the company Cabot;
dimethylsilyloxyl or polydimethylsiloxane groups, which are especially obtained by treating fumed silica in the presence of polydimethylsiloxane or dimethyldichlorosilane. Silicas thus treated are known as “Silica Dimethyl Silylate” according to the CTFA (8th Edition, 2000). They are sold, for example, under the references Aerosil R972® and Aerosil R974® by the company Degussa, and Cab-O-Sil TS-610® and Cab-O-Sil TS-720® by the company Cabot.

c) Silica aerogels are porous materials obtained by replacing (by drying) the liquid component of a silica gel with air.

The aerogels used according to the present invention are more particularly hydrophobic silica aerogels, preferably silyl silica aerogels (INCI name: Silica Silylate). “Hydrophobic silica” means any silica, the surface of which is treated with silylating agents, for example with halogenated silanes, such as alkylchlorosilanes, siloxanes, in particular dimethylsiloxanes, such as hexamethyldisiloxane, or silazanes, so as to functionalize the OH groups with silyl groups Si-Rn, for example trimethylsilyl groups.

As regards the preparation of hydrophobic silica aerogel particles that have been surface-modified by silylation, reference may be made to document U.S. Pat. No. 7,470,725.

Use will preferably be made of hydrophobic silica aerogel particles surface-modified with trimethylsilyl groups, preferably of the INCI name Silica Silylate.

As hydrophobic silica aerogels that may be used in the invention, an example that may be mentioned is the aerogel sold under the name VM-2260 or VM-2270 (INCI name: Silica Silylate) by the company Dow Corning, the particles of which have a mean size of about 1000 microns and a specific surface area per unit of mass ranging from 600 to 800 m²/g.

Mention may also be made of the aerogels sold by the company Cabot under the references Aerogel TLD 201, Aerogel OGD 201, Aerogel TLD 203, Enova® Aerogel MT 1100 and Enova Aerogel MT 1200.

As representatives of the lipophilic polymeric gelling agents that are suitable for use in the invention, mention may be made most particularly of:

a) The hydrocarbon-based block copolymers of the invention are preferably soluble or dispersible in the oily phase.

Such hydrocarbon-based block copolymers are described in patent application US-A-2002/005562 and in patent U.S. Pat. No. 5,221,534.

Advantageously, the hydrocarbon-based block copolymer is an amorphous block copolymer of styrene and olefin.

Block copolymers comprising at least one styrene block and at least one block comprising units chosen from butadiene, ethylene, propylene, butylene and isoprene or a mixture thereof are especially preferred.

According to one preferred embodiment, the hydrocarbon-based block copolymer is hydrogenated to reduce the residual ethylenic unsaturations after the polymerization of the monomers.

In particular, the hydrocarbon-based block copolymer is an optionally hydrogenated copolymer, containing styrene blocks and ethylene/C₃-C₄ alkylene blocks.

According to one preferred embodiment, the composition according to the invention comprises at least one diblock copolymer, which is preferably hydrogenated, preferably chosen from styrene-ethylene/propylene copolymers, styrene-ethylene/butadiene copolymers and styrene-ethylene/butylene copolymers. Diblock polymers are especially sold under the name Kraton® G1701E by the company Kraton Polymers.

According to another preferred embodiment, the composition according to the invention comprises at least one triblock copolymer, which is preferably hydrogenated, preferably chosen from styrene-ethylene/propylene-styrene copolymers, styrene-ethylene/butadiene-styrene copolymers, styrene-isoprene-styrene copolymers and styrene-butadiene-styrene copolymers. Triblock polymers are especially sold under the names Kraton® G1650, Kraton® D1101, Kraton® D1102 and Kraton® D1160 by the company Kraton Polymers.

According to one embodiment of the present invention, the hydrocarbon-based block copolymer is a styrene-ethylene/butylene-styrene triblock copolymer.

According to one preferred embodiment of the invention, it is especially possible to use a mixture of a styrene-butylene/ethylene-styrene triblock copolymer and of a styrene-ethylene/butylene diblock copolymer, especially the products sold under the name Kraton® G1657M by the company Kraton Polymers.

According to another preferred embodiment, the composition according to the invention comprises a mixture of styrene-butylene/ethylene-styrene hydrogenated triblock copolymer and of ethylene-propylene-styrene hydrogenated star polymer, such a mixture possibly being especially in isododecane or in another oil. Such mixtures are sold, for example, by the company Penreco under the trade names Versagel® M5960 and Versagel® M5670.

The hydrocarbon-based block copolymer(s) may be present in a content ranging from 0.5% to 15% by weight, preferably ranging from 1% to 10% by weight and even more advantageously from 2% to 8% by weight, relative to the total weight of the composition.

b) The polymers containing hydrogen bonding that are suitable for use in the invention may most particularly be polyamides and in particular hydrocarbon-based polyamides and silicone polyamides.

According to a particular mode, the polyamide used is an amide-terminated polyamide of formula (Ia):

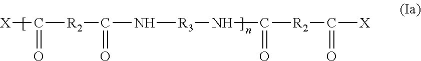

in which X represents a group —N(R₁)₂ in which R₁ is a linear or branched C₈ to C₂₂ alkyl radical which may be identical or different in each case, R₂ is a C₂₈-C₄₂ diacid dimer residue, R₃ is an ethylenediamine radical and n is between 2 and 5;

and mixtures thereof.

The oily phase of a composition according to the invention may also comprise, additionally in this case, at least one additional polyamide of formula (Ib):

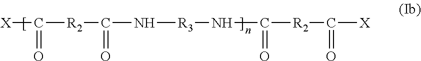

in which X represents a group —OR₁ in which R₁ is a linear or branched C₈ to C₂₂ and preferably C₁₆ to C₂₂ alkyl radical which may be identical or different in each case, R₂ is a C₂₈-C₄₂ diacid dimer residue, R₃ is an ethylenediamine radical and n is between 2 and 5, such as the commercial products sold by the company Arizona Chemical under the names Uniclear 80 and Uniclear 100 or Uniclear 80 V, Uniclear 100 V and Uniclear 100 VG, the INCI name of which is Ethylenediamine/Stearyl Dimer Dilinoleate Copolymer.

The silicone polyamides may preferentially be polymers comprising at least one unit of formula (III) or (IV):

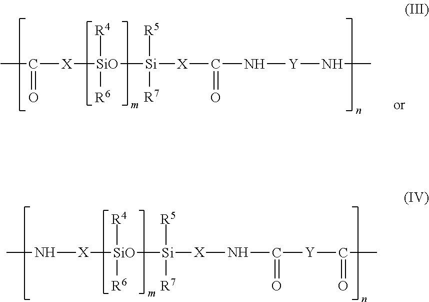

in which:
R⁴, R⁵, R⁶ and R⁷, which may be identical or different, represent a group chosen from:
   saturated or unsaturated, C₁ to C₄₀ linear, branched or cyclic hydrocarbon-based groups, which may contain in their chain one or more oxygen, sulfur and/or nitrogen atoms, and which may be partially or totally substituted with fluorine atoms,
   C₆ to C₁₀ aryl groups, optionally substituted with one or more C₁ to C₄ alkyl groups,
   polyorganosiloxane chains possibly containing one or more oxygen, sulfur and/or nitrogen atoms,
the groups X, which may be identical or different, represent a linear or branched C₁ to C₃₀ alkylenediyl group, possibly containing in its chain one or more oxygen and/or nitrogen atoms,
Y is a saturated or unsaturated C₁ to C₅₀ linear or branched alkylene, arylene, cycloalkylene, alkylarylene or arylalkylene divalent group, which may comprise one or more oxygen, sulfur and/or nitrogen atoms, and/or may bear as substituent one of the following atoms or groups of atoms: fluorine, hydroxyl, C₃ to C₈ cycloalkyl, C₁ to C₄₀ alkyl, C₅ to C₁₀ aryl, phenyl optionally substituted with 1 to 3 C₁ to C₃ alkyl, C₁ to C₃ hydroxyalkyl and C₁ to C₆ aminoalkyl groups, or
Y represents a group corresponding to the formula:

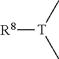

in which:
   T represents a linear or branched, saturated or unsaturated, C₃ to C₂₄ trivalent or tetravalent hydrocarbon-based group optionally substituted with a polyorganosiloxane chain, and possibly containing one or more atoms chosen from O, N and S, or T represents a trivalent atom chosen from N, P and Al, and
   R⁸ represents a linear or branched C₁ to C₅₀ alkyl group or a polyorganosiloxane chain, possibly comprising one or more ester, amide, urethane, thiocarbamate, urea, thiourea and/or sulfonamide groups, which may possibly be linked to another chain of the polymer,
n is an integer ranging from 2 to 500 and preferably from 2 to 200, and m is an integer ranging from 1 to 1000, preferably from 1 to 700 and better still from 6 to 200.

As an example of silicone polymers that may be used, mention may be made of one of the silicone polyamides obtained in accordance with examples 1 to 3 of document U.S. Pat. No. 5,981,680.

Mention may be made of the compounds sold by the company Dow Corning under the names DC 2-8179 (DP 100) and DC 2-8178 (DP 15), the INCI name of which is Nylon-611/Dimethicone Copolymers, i.e. Nylon-611/dimethicone copolymers. The silicone polymers and/or copolymers advantageously have a temperature of transition from the solid state to the liquid state ranging from 45° C. to 190° C. Preferably, they have a temperature of transition from the solid state to the liquid state ranging from 70° C. to 130° C. and better still from 80° C. to 105° C.

c) The term “organopolysiloxane elastomer” or “silicone elastomer” means a supple, deformable organopolysiloxane with viscoelastic properties and especially with the consistency of a sponge or a supple sphere. It is more particularly a crosslinked organopolysiloxane elastomer. The elastomer is advantageously a non-emulsifying elastomer, i.e. an organopolysiloxane elastomer not containing any hydrophilic chains, and in particular not containing any polyoxyalkylene units (especially polyoxyethylene or polyoxypropylene) or any polyglyceryl units.

In particular, the silicone elastomer used in the present invention may be chosen from Dimethicone Crosspolymer (INCI name), Vinyl Dimethicone Crosspolymer (INCI name), Dimethicone/Vinyl Dimethicone Crosspolymer (INCI name), Dimethicone Crosspolymer-3 (INCI name).

The organopolysiloxane elastomer particles may be conveyed in the form of a gel constituted of an elastomeric organopolysiloxane included in at least one hydrocarbon-based oil and/or one silicone oil. Non-emulsifying elastomers are especially described in patents EP 242 219, EP 285 886 and EP 765 656 and in patent application JP-A-61-194 009.

Mention may be made especially of the compounds having the following INCI names:
Dimethicone/Vinyl Dimethicone Crosspolymer, such as USG-105 and USG-107A from the company Shin-Etsu; DC9506 and DC9701 from the company Dow Corning;
Dimethicone/Vinyl Dimethicone Crosspolymer (and) Dimethicone, such as KSG-6 and KSG-16 from the company Shin-Etsu;
Dimethicone/Vinyl Dimethicone Crosspolymer (and) Cyclopentasiloxane, such as KSG-15;
Cyclopentasiloxane (and) Dimethicone Crosspolymer, such as DC9040, DC9045 and DC5930 from the company Dow Corning;
Dimethicone (and) Dimethicone Crosspolymer, such as DC9041 from the company Dow Corning;
Dimethicone (and) Dimethicone Crosspolymer, such as Dow Corning EL-9240® Silicone Elastomer Blend from the company Dow Corning (mixture of polydimethylsiloxane crosslinked with hexadiene/polydimethylsiloxane (2 cSt));
C₄₋₂₄ Alkyl Dimethicone/Divinyl Dimethicone Crosspolymer, such as NuLastic Silk MA from the company Alzo;
Dimethicone/Vinyl Dimethicone Crosspolymer (and) Dimethicone, such as KSG-6 and KSG-16 from the company Shin-Etsu;
Dimethicone (and) Dimethicone Crosspolymer, such as DC9041 from the company Dow Corning;
Dimethicone (and) Dimethicone Crosspolymer, such as Dow Corning EL-9240® Silicone Elastomer Blend from the company Dow Corning (mixture of polydimethylsiloxane crosslinked with hexadiene/polydimethylsiloxane (2 cSt)); and
the mixture of polydimethylsiloxane crosslinked with hexadiene/polydimethylsiloxane (5 cSt) sold under the name DC 9041 by Dow Corning.

The organopolysiloxane elastomer particles may also be used in powder form: mention may be made especially of the powders sold under the names Dow Corning 9505 Powder and Dow Corning 9506 Powder by the company Dow Corning, these powders having the INCI name: Dimethicone/Vinyl Dimethicone Crosspolymer. The organopolysiloxane powder may also be coated with silsesquioxane resin, as described, for example, in patent U.S. Pat. No. 5,538,793. Such elastomeric powders are sold under the names KSP-100, KSP-101, KSP-102, KSP-103, KSP-104 and KSP-105 by the company Shin-Etsu, and have the INCI name: vinyl Dimethicone/Methicone Silsesquioxane Crosspolymer.

d) For the purposes of the invention, the term “semicrystalline polymer” means polymers comprising a crystallizable portion and an amorphous portion and having a first-order reversible change of phase temperature, in particular of melting point (solid-liquid transition). The crystallizable part is either a side chain (or pendent chain) or a block in the backbone. Preferably, the semicrystalline polymers of the invention are of synthetic origin. According to one preferred embodiment, the semicrystalline polymer is chosen from:
homopolymers and copolymers comprising units resulting from the polymerization of one or more monomers bearing crystallizable hydrophobic side chain(s),
polymers bearing in the backbone at least one crystallizable block,
polycondensates of aliphatic or aromatic or aliphatic/aromatic polyester type,
copolymers of ethylene and propylene prepared via metallocene catalysis, and
acrylate/silicone copolymers.

The semicrystalline polymers that may be used in the invention may be chosen in particular from:
block copolymers of polyolefins of controlled crystallization, whose monomers are described in EP 0 951 897,
polycondensates, in particular of aliphatic or aromatic or aliphatic/aromatic polyester type,
copolymers of ethylene and propylene prepared via metallocene catalysis,
homopolymers or copolymers bearing at least one crystallizable side chain and homopolymers or copolymers bearing in the backbone at least one crystallizable block, such as those described in document U.S. Pat. No. 5,156,911, such as the (C₁₀-C₃₀)alkyl polyacrylates corresponding to the Intelimer® products from the company Landec described in the brochure Intelimer® Polymers, Landec IP22 (Rev. 4-97), for example the product Intelimer® IPA 13-1 from the company Landec, which is a polystearyl acrylate with a molecular weight of about 145 000 and a melting point of 49° C.,
homopolymers or copolymers bearing at least one crystallizable side chain, in particular containing fluoro group(s), as described in document WO 01/19333,
acrylate/silicone copolymers, such as copolymers of acrylic acid and of stearyl acrylate bearing polydimethylsiloxane grafts, copolymers of stearyl methacrylate bearing polydimethylsiloxane grafts, copolymers of acrylic acid and of stearyl methacrylate bearing polydimethylsiloxane grafts, copolymers of methyl methacrylate, butyl methacrylate, 2-ethylhexyl acrylate and stearyl methacrylate bearing polydimethylsiloxane grafts. Mention may be made in particular of the copolymers sold by the company Shin-Etsu under the names KP-561 (CTFA name: Acrylates/Dimethicone), KP-541 (CTFA name: Acrylates/Dimethicone and Isopropyl Alcohol), KP-545 (CTFA name: Acrylates/Dimethicone and Cyclopentasiloxane),

and mixtures thereof.

The polymeric gelling agent may also be a dextrin ester. It is preferably at least one C₁₂ to C₂₄ and in particular C₁₄ to C₁₈ fatty acid and dextrin ester, or mixtures thereof. Preferably, the dextrin ester is chosen from dextrin myristate and/or dextrin palmitate, and mixtures thereof. According to a preferred embodiment, this product may be chosen, for example, from those sold under the names Rheopearl TL®, Rheopearl KL® and Rheopearl® KL2 by the company Chiba Flour Milling and Rheopearl MKL-2 by the company Chiba Flour Milling.

According to one embodiment, a composition according to the invention comprises at least one additional ingredient, said additional ingredient being at least:
a hydrophilic gelling agent chosen from fumed silicas, synthetic polymeric gelling agents, polymeric gelling agents that are natural or of natural origin, for instance non-starchy polysaccharides, native starches, modified starches, starches modified with an acrylic polymer, hydrolyzed starches grafted with an acrylic polymer, polymers based on starch, gum and cellulose derivative, acrylic or methacrylic acid homopolymers or copolymers, salts thereof or esters thereof, polyacrylic acid/alkyl acrylate copolymers, crosslinked or non-crosslinked polyoxyethylenated AMPS/alkyl methacrylate copolymers, anionic, cationic, amphoteric or nonionic chitin or chitosan polymers, cellulose polymers, other than alkylcellulose, chosen from hydroxyethylcellulose, hydroxypropylcellulose, hydroxymethylcellulose, ethylhydroxyethylcellulose, carboxymethylcellulose, and also quaternized cellulose derivatives, vinyl polymers and anionic, cationic, nonionic or amphoteric associative polymers, modified or unmodified polymers of natural origin, alginates and carrageenans, glycoaminoglycans, hyaluronic acids and derivatives thereof, deoxyribonucleic acid, mucopolysaccharides, and chondroitin sulfates, and mixtures thereof; and/or
a lipophilic gelling agent chosen from particulate gelling agents, organopolysiloxane elastomers, semicrystalline polymers, hydrocarbon-based block copolymers, dextrin esters and polymers bearing hydrogen bonding, and mixtures thereof.

A composition according to the invention may advantageously comprise from 0.01% to 8% by weight and preferably from 0.1% to 3% by weight of gelling agent(s) relative to the total weight of the composition.

Hydrophobic Film-Forming Polymers

A composition according to the invention may also comprise as additional ingredient at least one hydrophobic film-forming polymer.

For the purposes of the present invention, the term “hydrophobic film-forming polymer” is intended to denote a film-forming polymer that has no affinity for water and, in this respect, does not lend itself to a formulation in the form of a solute in an aqueous medium. In particular, the term “hydrophobic polymer” means a polymer having a solubility in water at 25° C. of less than 1% by weight.

The term “film-forming” polymer means a polymer that is capable of forming, by itself or in the presence of an auxiliary film-forming agent, a macroscopically continuous deposit on a support, especially on the skin and/or the nails, and preferably a cohesive deposit, and better still a deposit whose cohesion and mechanical properties are such that said deposit can be isolable and manipulable in isolation, for example when said deposit is prepared by pouring onto a non-stick surface, such as a Teflon-coated or silicone-coated surface.

In particular, the hydrophobic film-forming polymer is a polymer chosen from the group comprising:
film-forming polymers that are soluble in an organic solvent medium, in particular liposoluble polymers; this means that the polymer is soluble or miscible in the organic medium and forms a single homogeneous phase when it is incorporated into the medium; and
film-forming polymers that are dispersible in an organic solvent medium, which means that the polymer forms an insoluble phase in the organic medium, the polymer remaining stable and/or compatible once incorporated into this medium. In particular, such polymers may be in the form of non-aqueous dispersions of polymer particles, preferably dispersions in silicone oils or hydrocarbon-based oils; in one embodiment, the non-aqueous polymer dispersions comprise polymer particles stabilized on their surface with at least one stabilizer; these non-aqueous dispersions are often referred to as NADs.

Hydrophobic film-forming polymers that may especially be mentioned include homopolymers and copolymers of a compound bearing an ethylenic unit, acrylic polymers and copolymers, polyurethanes, polyesters, silicone polymers, such as polymers bearing a non-silicone organic backbone grafted with monomers containing a polysiloxane, or polyisoprenes.

A composition according to the invention may comprise from 1% to 30% by weight, preferably from 2% to 25% by weight and even more preferentially from 5% to 20% by weight of hydrophobic film-forming polymer(s) relative to the total weight of the composition.

As hydrophobic film-forming polymers that are most particularly suitable for use in the invention, mention may be made especially of lipodispersible film-forming polymers in the form of non-aqueous dispersions of polymer particles (NADs), block ethylenic copolymers, vinyl polymers comprising at least one unit derived from carbosiloxane dendrimer, silicone acrylate copolymers and mixtures thereof, preferably lipodispersible film-forming polymers in the form of non-aqueous dispersions of polymer particles (NADs).

1. Lipodispersible Film-Forming Polymers in the Form of Non-Aqueous Dispersions of Polymer Particles, Also Known as NADs

According to another embodiment variant, a composition according to the invention may comprise, as hydrophobic film-forming polymer, at least one polymer chosen from lipodispersible film-forming polymers in the form of non-aqueous dispersions of polymer particles, also known as NADs.

Use may be made, as non-aqueous dispersion of hydrophobic film-forming polymer, of dispersions of particles of a grafted ethylenic polymer, preferably an acrylic polymer, in a liquid oily phase, for example in the form of surface-stabilized particles dispersed in the liquid fatty phase.

The dispersion of surface-stabilized polymer particles can be manufactured as described in the document WO 04/055081.

2. Block Ethylenic Copolymer

According to a first embodiment of the invention, the hydrophobic film-forming polymer is a block ethylenic copolymer, containing at least a first block with a glass transition temperature (T_{g}) of greater than or equal to 40° C. which is totally or partly derived from one or more first monomers, which are such that the homopolymer prepared from these monomers has a glass transition temperature of greater than or equal to 40° C., and at least a second block with a glass transition temperature of less than or equal to 20° C. which is derived totally or partly from one or more second monomers, which are such that the homopolymer prepared from these monomers has a glass transition temperature of less than or equal to 20° C., said first block and said second block being connected together via a statistical intermediate segment comprising at least one of said first constituent monomers of the first block and at least one of said second constituent monomers of the second block, and said block copolymer having a polydispersity index I of greater than 2.

Polymers of this type that are suitable for use in the invention are described in document EP 1 411 069.

As examples of such polymers, mention may be made more particularly of Mexomere PAS® (acrylic acid/isobutyl acrylate/isobornyl acrylate copolymer 50% diluted in isododecane) sold by the company Chimex.

3. Vinyl Polymer Comprising at Least One Carbosiloxane Dendrimer-Based Unit

According to one particular embodiment, a composition used according to the invention may comprise, as hydrophobic film-forming polymer, at least one vinyl polymer comprising at least one carbosiloxane dendrimer-based unit.

The vinyl polymer used according to the invention especially has a backbone and at least one side chain, which comprises a carbosiloxane dendrimer-based unit having a carbosiloxane dendrimer structure.

Vinyl polymers comprising at least one carbosiloxane dendrimer unit as described in patent applications WO 03/045 337 and EP 963 751 by the company Dow Corning may be used in particular.

In the context of the present invention, the term “carbosiloxane dendrimer structure” represents a molecular structure containing branched groups of high molecular masses, said structure having high regularity in the radial direction starting from the bond to the backbone. Such carbosiloxane dendrimer structures are described in the form of a highly branched siloxane-silylalkylene copolymer in the laid-open Japanese patent application Kokai 9-171 154.

A vinyl polymer containing at least one carbosiloxane dendrimer-based unit has a molecular side chain containing a carbosiloxane dendrimer structure, and may be derived from the polymerization of:

(A) from 0 to 99.9 parts by weight of a vinyl monomer; and

(B) from 100 to 0.1 part by weight of a carbosiloxane dendrimer containing a radical-polymerizable organic group, represented by the general formula:

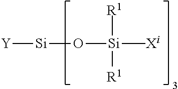

in which Y represents a radical-polymerizable organic group, le represents an aryl group or an alkyl group containing from 1 to 10 carbon atoms, and Xⁱ represents a silylalkyl group which, when i=1, is represented by the formula:

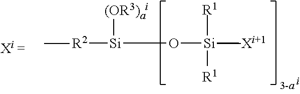

in which R¹ is as defined above, R² represents an alkylene group containing from 2 to 10 carbon atoms, R³ represents an alkyl group containing from 1 to 10 carbon atoms, X′⁺¹ represents a hydrogen atom, an alkyl group containing from 1 to 10 carbon atoms, an aryl group, or the silylalkyl group defined above with i=i+1; i is an integer from 1 to 10 which represents the generation of said silylalkyl group, and a′ is an integer from 0 to 3;

in which said radical-polymerizable organic group contained in component (A) is chosen from:
organic groups containing a methacrylic group or an acrylic group and that are represented by the formulae:

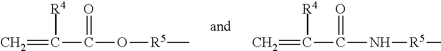

in which R⁴ represents a hydrogen atom or an alkyl group, R⁵ represents an alkylene group containing from 1 to 10 carbon atoms; and
organic groups containing a styryl group and that are represented by the formula:

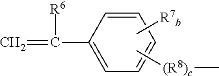

in which R⁶ represents a hydrogen atom or an alkyl group, R⁷ represents an alkyl group containing from 1 to 10 carbon atoms, R⁸ represents an alkylene group containing from 1 to 10 carbon atoms, b is an integer from 0 to 4, and c is 0 or 1, such that if c is 0, —(R⁸)_{c}-represents a bond.

The monomer of vinyl type that is component (A) in the vinyl polymer is a monomer of vinyl type that contains a radical-polymerizable vinyl group.

There is no particular limitation as regards such a monomer.

The following are examples of this monomer of vinyl type: methyl methacrylate, ethyl methacrylate, n-propyl methacrylate, isopropyl methacrylate or a methacrylate of an analogous lower alkyl; glycidyl methacrylate; butyl methacrylate, butyl acrylate, n-butyl methacrylate, isobutyl methacrylate, tert-butyl acrylate, tert-butyl methacrylate, n-hexyl methacrylate, cyclohexyl methacrylate, 2-ethylhexyl acrylate, 2-ethylhexyl methacrylate, octyl methacrylate, lauryl methacrylate, stearyl acrylate, stearyl methacrylate or an analogous higher methacrylate; vinyl acetate, vinyl propionate or a vinyl ester of an analogous lower fatty acid; vinyl caproate, vinyl 2-ethylhexoate, vinyl laurate, vinyl stearate or an ester of an analogous higher fatty acid; styrene, vinyltoluene, benzyl methacrylate, phenoxyethyl methacrylate, vinylpyrrolidone or analogous vinyl aromatic monomers; methacrylamide, N-methylolmethacrylamide, N-methoxymethylmethacrylamide, isobutoxymethoxymethacrylamide, N,N-dimethylmethacrylamide or analogous monomers of vinyl type containing amide groups; hydroxyethyl methacrylate, hydroxypropyl alcohol methacrylate or analogous monomers of vinyl type containing hydroxyl groups; acrylic acid, methacrylic acid, itaconic acid, crotonic acid, fumaric acid, maleic acid or analogous monomers of vinyl type containing a carboxylic acid group; tetrahydrofurfuryl methacrylate, butoxyethyl methacrylate, ethoxydiethylene glycol methacrylate, polyethylene glycol methacrylate, polypropylene glycol monomethacrylate, hydroxybutyl vinyl ether, cetyl vinyl ether, 2-ethylhexyl vinyl ether or an analogous monomer of vinyl type with ether bonds; methacryloxypropyltrimethoxysilane, polydimethylsiloxane containing a methacrylic group on one of its molecular ends, polydimethylsiloxane containing a styryl group on one of its molecular ends, or an analogous silicone compound containing unsaturated groups; butadiene; vinyl chloride; vinylidene chloride; methacrylonitrile; dibutyl fumarate; anhydrous maleic acid; anhydrous succinic acid; methacryl glycidyl ether; an organic salt of an amine, an ammonium salt, and an alkali metal salt of methacrylic acid, of itaconic acid, of crotonic acid, of maleic acid or of fumaric acid; a radical-polymerizable unsaturated monomer containing a sulfonic acid group such as a styrenesulfonic acid group; a quaternary ammonium salt derived from methacrylic acid, such as 2-hydroxy-3-methacryloxypropyltrimethylammonium chloride; and a methacrylic acid ester of an alcohol containing a tertiary amine group, such as a methacrylic acid ester of diethylamine.

Multifunctional monomers of vinyl type can also be used.

The following are examples of such compounds: trimethylolpropane trimethacrylate, pentaerythrityl trimethacrylate, ethylene glycol dimethacrylate, tetraethylene glycol dimethacrylate, polyethylene glycol dimethacrylate, 1,4-butanediol dimethacrylate, 1,6-hexanediol dimethacrylate, neopentyl glycol dimethacrylate, trimethylolpropane trioxyethylmethacrylate, tris(2-hydroxyethyl) isocyanurate dimethacrylate, tris(2-hydroxyethyl) isocyanurate trimethacrylate, polydimethylsiloxane capped with styryl groups bearing divinylbenzene groups on the two ends, or analogous silicone compounds bearing unsaturated groups.

To facilitate the preparation of a starting material mixture for cosmetic products, the number-average molecular mass of the vinyl polymer which contains a carbosiloxane dendrimer may be chosen within the range between 3000 g/mol and 2 000 000 g/mol and preferably between 5000 g/mol and 800 000 g/mol. It may be a liquid, a gum, a paste, a solid, a powder or any other form. The preferred forms are solutions formed by dilution of a dispersion or of a powder in solvents such as a silicone oil or an organic oil.

A vinyl polymer contained in the dispersion or the solution may have a concentration in the range between 0.1% and 95% by weight and preferably between 5% and 70% by weight. However, to facilitate the handling and the preparation of the mixture, the range should preferably be between 10% and 60% by weight.

According to one preferred mode, a vinyl polymer that is suitable for use in the invention may be one of the polymers described in the examples of patent application EP 0 963 751.

According to a preferred embodiment, a vinyl polymer grafted with a carbosiloxane dendrimer may be the product of polymerization of:

(A) from 0.1 to 99 parts by weight of one or more acrylate or methacrylate monomers; and

(B) from 100 to 0.1 part by weight of an acrylate or methacrylate monomer of a tris[tri(trimethylsiloxy)silylethyldimethylsiloxy]silylpropyl carbosiloxane dendrimer.

According to one embodiment, a vinyl polymer bearing at least one carbosiloxane dendrimer-based unit may comprise a tris[tri(trimethylsiloxy)silylethyldimethylsiloxy]silylpropyl carbosiloxane dendrimer-based unit corresponding to one of the formulae:

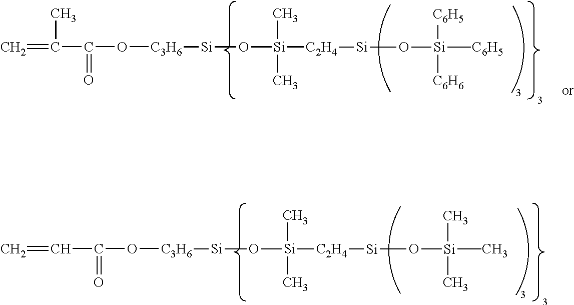

According to one preferred mode, a vinyl polymer bearing at least one carbosiloxane dendrimer-based unit used in the invention comprises at least one butyl acrylate monomer.

According to one embodiment, a vinyl polymer may also comprise at least one fluorinated organic group. A fluorinated vinyl polymer can be one of the polymers described in the examples of application WO 03/045337.

According to one preferred embodiment, a vinyl polymer grafted in the sense of the present invention may be conveyed in an oil or a mixture of oils, which is/are in particular preferably volatile, chosen from silicone oils and hydrocarbon-based oils, and mixtures thereof.

According to a particular embodiment, a silicone oil that is suitable for use in the invention may be cyclopentasiloxane or cyclohexasiloxane.

According to another particular embodiment, a hydrocarbon-based oil that is suitable for use in the invention may be isododecane.

Vinyl polymers grafted with at least one carbosiloxane dendrimer-based unit that may be particularly suitable for use in the present invention are the polymers sold under the names TIB 4-100, TIB 4-101, TIB 4-120, TIB 4-130, TIB 4-200, FA 4002 ID (TIB 4-202), TIB 4-220 and FA 4001 CM (TIB 4-230) by the company Dow Corning. Use will preferably be made of the polymers sold under the names FA 4002 ID (TIB 4-202) and FA 4001 CM (TIB 4-230) by Dow Corning.

Preferably, the vinyl polymer grafted with at least one carbosiloxane dendrimer-based unit that may be used in a composition of the invention is an acrylate/polytrimethyl siloxymethacrylate copolymer, especially the product sold in isododecane under the name Dow Corning FA 4002 ID Silicone Acrylate by the company Dow Corning.

4. Silicone Acrylate Copolymers

According to a particular embodiment, a composition used according to the invention may comprise, as hydrophobic film-forming polymer, at least one copolymer comprising carboxylate groups and polydimethylsiloxane groups.

In the present patent application, the term “copolymer comprising carboxylate groups and polydimethylsiloxane groups” means a copolymer obtained from (a) one or more carboxylic (acid or ester) monomers, and (b) one or more polydimethylsiloxane (PDMS) chains.

In the present patent application, the term “carboxylic monomer” means both carboxylic acid monomers and carboxylic acid ester monomers. Thus, the monomer (a) may be chosen, for example, from acrylic acid, methacrylic acid, maleic acid, fumaric acid, itaconic acid and crotonic acid, esters thereof and mixtures of these monomers. Mention may be made, as esters, of the following monomers: acrylate, methacrylate, maleate, fumarate, itaconate and/or crotonate. According to a preferred embodiment of the invention, the monomers in ester form are more particularly chosen from linear or branched, preferably C₁-C₂₄ and better still C₁-C₂₂ alkyl acrylates and methacrylates, the alkyl radical preferably being chosen from methyl, ethyl, stearyl, butyl and 2-ethylhexyl radicals, and mixtures thereof.

Thus, according to a particular embodiment of the invention, the copolymer comprises as carboxylate groups at least one group chosen from acrylic acid and methacrylic acid, and methyl, ethyl, stearyl, butyl or 2-ethylhexyl acrylates or methacrylates, and mixtures thereof.

In the present patent application, the term “polydimethylsiloxanes” (also known as organopolysiloxanes and abbreviated as PDMS) denotes, in accordance with what is generally accepted, any organosilicon polymer or oligomer of linear structure, of variable molecular weight, obtained by polymerization and/or polycondensation of suitably functionalized silanes, and consisting essentially of a repetition of main units in which the silicon atoms are linked together via oxygen atoms (siloxane bond comprising trimethyl radicals directly linked via a carbon atom to said silicon atoms. The PDMS chains that may be used to obtain the copolymer used according to the invention comprise at least one polymerizable radical group, preferably located on at least one of the ends of the chain, i.e. the PDMS may contain, for example, a polymerizable radical group on the two ends of the chain or one polymerizable radical group on one end of the chain and one trimethylsilyl end group on the other end of the chain. The polymerizable radical group may especially be an acrylic or methacrylic group, in particular a CH₂═CR₁—CO—O— R₂ group, in which R₁ represents a hydrogen or a methyl group and R₂ represents —CH₂—, —(CH₂)ₙ with n=3, 5, 8 or 10, —CH₂—CH(CH₃)—CH₂—, —CH₂—CH₂—O—CH₂—CH₂—, —CH₂—CH₂—O—CH₂—CH₂—CH(CH₃)—CH₂—, —CH₂—CH₂—CH₂ CH₂— CH₂—CH₂—CH₂—.

The copolymers used in the composition of the invention are generally obtained according to the usual methods of polymerization and grafting, for example by free-radical polymerization (A) of a PDMS comprising at least one polymerizable radical group (for example on one of the ends of the chain or on both ends) and (B) of at least one carboxylic monomer, as described, for example, in documents U.S. Pat. No. 5,061,481 and U.S. Pat. No. 5,219,560.

The copolymers obtained generally have a molecular weight ranging from approximately 3000 g/mol to 200 000 g/mol and preferably from approximately 5000 g/mol to 100 000 g/mol.

The copolymer used in the composition of the invention may be in its native form or in dispersed form in a solvent such as lower alcohols containing from 2 to 8 carbon atoms, for instance isopropyl alcohol, or oils, for instance volatile silicone oils (for example cyclopentasiloxane).

As copolymers that may be used in the composition of the invention, mention may be made, for example, of copolymers of acrylic acid and of stearyl acrylate bearing polydimethylsiloxane grafts, copolymers of stearyl methacrylate bearing polydimethylsiloxane grafts, copolymers of acrylic acid and of stearyl methacrylate bearing polydimethylsiloxane grafts, copolymers of methyl methacrylate, butyl methacrylate, 2-ethylhexyl acrylate and stearyl methacrylate bearing polydimethylsiloxane grafts. As copolymers that may be used in the composition of the invention, mention may be made in particular of the copolymers sold by the company Shin-Etsu under the names KP-561 (CTFA name: Acrylates/Dimethicone), KP-541 in which the copolymer is dispersed at 60% by weight in isopropyl alcohol (CTFA name: Acrylates/Dimethicone and Isopropyl Alcohol), and KP-545 in which the copolymer is dispersed at 30% in cyclopentasiloxane (CTFA name: Acrylates/Dimethicone and Cyclopentasiloxane). According to one preferred embodiment of the invention, KP561 is preferably used; this copolymer is not dispersed in a solvent, but is in waxy form, its melting point being about 30° C.

Mention may also be made of the grafted copolymer of polyacrylic acid and dimethylpolysiloxane dissolved in isododecane, sold by the company Shin-Etsu under the name KP-550.

Ingredients Chosen from Cosmetic Active Agents, Fragrances and Dyestuffs

According to one embodiment, the additional ingredient included in a composition according to the invention is at least one compound chosen from cosmetic active agents; vitamins; UV-screening agents; dyestuffs, preferably chosen from pigments and nacres; fragrances; and mixtures thereof.

Cosmetic Active Agents

A cosmetic active agent according to the present invention may be chosen especially from moisturizers (also known as wetting agents), UV-screening agents, cicatrizing agents, bleaching or depigmenting agents, antiperspirant agents and/or anti-aging agents. Certain anionic surfactants used in products for cleansing the skin and/or the nails, and especially facial and/or bodily skin, may also be considered as cosmetic active agents.

As moisturizers, mention may be made especially of polyols; ceramides; DHEA and derivatives thereof; coenzyme Q10; and hyaluronic acid and derivatives thereof.

As examples of cicatrizing agents, mention may be made especially of allantoin, urea, and certain amino acids such as hydroxyproline, arginine and serine.

As examples of antiperspirant agents, mention may be made of astringent salts, for instance aluminum salts and/or zirconium salts.

As examples of anti-aging agents, mention may be made especially of keratolytic or pro-desquamating agents, for example α-hydroxy acids, β-hydroxy acids, α-keto acids, β-keto acids, retinoids and esters thereof, retinal, and retinoic acid and derivatives thereof.

As ionic surfactants that are suitable for use as cosmetic active agents, mention may be made especially of anionic surfactants chosen from alkyl sulfates; alkyl ether sulfates; acylglutamates; acyl sarcosinates; acyllactylates; alkyl ether carboxylates; acylisethionates such as cocoyl isethionates and/or lauroyl isethionates.

According to a particular implementation variant, a composition according to the invention contains at least, as ingredient other than the synthetic phyllosilicate, crosslinked and/or non-crosslinked hyaluronic acid, or a derivative thereof. The term “hyaluronic acid derivative” preferentially means a salt thereof, in particular the sodium salt thereof. It is preferably sodium hyaluronate (NaHA).

A composition according to the invention may thus advantageously comprise from 0.1% to 10% by weight and preferably from 0.5% to 5% by weight of hyaluronic acid or a derivative thereof relative to the total weight of the composition.

Preferably, a composition according to the invention may comprise from 0.001% to 30% by weight, preferably from 0.01% to 20% by weight, better still from 0.01% to 10% by weight, preferably ranging from 0.01% to 5% by weight, of cosmetic active agent(s) by weight relative to the total weight of the composition.

Fragrances

A composition according to the invention may also comprise a fragrance especially in the form of an essential oil or a mixture of essential oils.

Dyestuffs

The compositions in accordance with the invention advantageously comprise at least one dyestuff.

This (or these) dyestuff(s) are preferably chosen from pulverulent dyestuffs, liposoluble dyes and water-soluble dyes, and mixtures thereof.

Preferably, the compositions according to the invention comprise at least one pulverulent dyestuff. The pulverulent dyestuffs may be chosen from pigments and nacres, and preferably from pigments.

The pigments may be white or colored, mineral and/or organic, and coated or uncoated. Among the mineral pigments, mention may be made of metal oxides, in particular titanium dioxide, optionally surface-treated, zirconium, zinc or cerium oxides, and also iron, titanium or chromium oxides, manganese violet, ultramarine blue, chromium hydrate and ferric blue. Among the organic pigments that may be mentioned are carbon black, pigments of D & C type and lakes based on cochineal carmine or on barium, strontium, calcium or aluminum.

The nacres may be chosen from white nacreous pigments such as mica coated with titanium or with bismuth oxychloride, colored nacreous pigments such as titanium mica with iron oxides, titanium mica with in particular ferric blue or chromium oxide, titanium mica with an organic pigment of the abovementioned type, and also nacreous pigments based on bismuth oxychloride.

The liposoluble dyes are, for example, Sudan Red, D&C Red 17, D&C Green 6, (3-carotene, soybean oil, Sudan Brown, D&C Yellow 11, D&C Violet 2, D&C Orange 5, quinoline yellow and annatto.

Preferably, the pigments contained in the compositions according to the invention are chosen from metal oxides.

These dyestuffs may be present in a content ranging from 0.01% to 30% by weight relative to the total weight of the composition and in particular from 3% to 22% by weight relative to the total weight of the composition.

Preferably, the dyestuff(s) are chosen from one or more metal oxides that are present in a content of greater than or equal to 2% by weight relative to the total weight of the composition, and advantageously of inclusively between 3% and 25% by weight relative to the total weight of the composition.

Vitamins

A composition according to the invention may also comprise at least one vitamin chosen, for example, from vitamins A, B3, PP, B5, E, K1 and/or C and derivatives of these vitamins and especially the esters thereof, and mixtures thereof.

Preserving Agents

The term “preserving agent” is intended to denote a substance of natural or synthetic origin incorporated into a composition, especially a cosmetic composition, for the purpose of preventing chemical alterations (oxidation) or microbiological alterations.

The preserving agents according to the invention may be chosen from antimicrobial preserving agents, antioxidant preserving agents, and mixtures thereof. The choice of these compounds clearly falls within the competence of a person skilled in the art.

Needless to say, all the abovementioned additional agents or compounds are different from the synthetic phyllosilicates according to the invention.

Physiologically Acceptable Medium

As presented above, a composition according to the invention may advantageously be a cosmetic or dermatological composition.

In this particular embodiment, since a composition according to the invention is intended for topical application to the skin and/or the nails, it contains a physiologically acceptable medium.

For the purposes of the present invention, the term “physiologically acceptable medium” means a medium that is compatible with the skin and/or the nails.

Thus, the physiologically acceptable medium is in particular a cosmetically or dermatologically acceptable medium, i.e. a medium that has no unpleasant odor, color or appearance, and that does not cause the user any unacceptable stinging, tautness or redness.

Preferably, a composition of the invention is a cosmetic composition.

In the light the foregoing, a composition according to the invention is intended for topical administration, i.e. by application to the surface of the skin and/or the nails.

Thus, a composition according to the invention may be in the form of products for caring for the skin or semimucous membranes, such as a protective, treatment or care composition for the face, for the lips, for the hands, for the nails or even false nails, for the feet, for the anatomical folds or for the body (for example, day cream, night cream, makeup-removing cream, makeup base, anti-sun composition, protective or care body milk, aftersun milk, skincare lotion, gel or foam, serum, mask, artificial tanning composition, aftershave composition, hygiene and cleansing product for the skin and/or the nails).

The composition according to the invention may be in any presentation form normally used in cosmetics and dermatology.

It may especially be in the form of an optionally gelled aqueous or aqueous-alcoholic solution, an optionally two-phase or three-phase lotion-type dispersion, an oil-in-water or water-in-oil or multiple emulsion, an aqueous gel, or a dispersion of oils in an aqueous phase. It may also be of solid or pasty consistency.

According to a particular embodiment, a composition according to the invention may also advantageously be in a formulation said to be of gel-gel type, i.e. exemplified by a composition comprising:
at least one aqueous phase gelled with at least one hydrophilic gelling agent; and
at least one oily phase gelled with at least one lipophilic gelling agent, the two phases forming therein a macroscopically homogeneous mixture.

Such an embodiment is especially illustrated in example 4 below.

According to this particular embodiment, the aqueous or aqueous-alcoholic gel of synthetic phyllosilicate may be used as hydrophilic gelling agent. Specifically, by virtue of its specific properties, an aqueous or aqueous-alcoholic gel of synthetic phyllosilicate in accordance with the invention may act as hydrophilic gelling agent and/or filler in a composition according to the invention.

According to one embodiment, a composition of the invention is in the form of a composition for cleansing the skin and/or the nails, especially bodily or facial skin, in particular facial skin.

According to one embodiment, a composition of the invention may advantageously be in the form of a composition for caring for the skin and/or the nails, especially bodily or facial skin, in particular facial skin.

According to another embodiment, a composition of the invention may advantageously be in the form of a composition for making up the skin and/or the nails, especially bodily or facial skin, in particular facial skin.

Thus, according to a sub-mode of this embodiment, a composition of the invention may advantageously be in the form of a makeup base composition for makeup purposes.

A composition of the invention may advantageously be in the form of a foundation.

According to another sub-mode of this embodiment, a composition of the invention may advantageously be in the form of a composition for making up bodily or facial skin, and especially facial skin. It may thus be an eyeshadow or a face powder.

According to one embodiment, a composition according to the invention may be in the form of a composition for caring for and/or making up the skin and/or the nails, of the body or of the face, in particular of the face, and is preferably a foundation, a face powder or an eyeshadow.

According to yet another sub-mode of this embodiment, a composition of the invention may advantageously be in the form of a product intended for caring for and/or making up the lips and is preferably a stick, such as a lipstick, a varnish or a gloss. The synthetic phyllosilicate in aqueous gel form according to the invention has the advantage of leading to the formulation of emulsion sticks that may be used in the care and makeup sector.

According to yet another sub-mode of this embodiment, a composition of the invention may advantageously be in the form of a product intended for caring for and/or making up the nails, or false nails, and is preferably a varnish.

Such compositions are in particular prepared according to the general knowledge of those skilled in the art.

Processes and Uses

In all cases, a composition according to the invention may be prepared according to methods known to those skilled in the art. Needless to say, the process for preparing the compositions according to the invention depends on the desired type of formulation.

Since the synthetic phyllosilicates according to the invention are used in the form of an aqueous or aqueous-alcoholic gel, their incorporation is thus favored in the aqueous phase.

As indicated previously, the present invention relates to a process for making up and/or caring for the skin and/or the nails, comprising a step of applying to said skin and/or said nails a composition comprising, in a physiologically acceptable medium, at least one aqueous or aqueous-alcoholic gel of synthetic phyllosilicate of formula Mg₃Si₄O₁₀(OH)₂ in accordance with the invention.

According to one embodiment, the composition used is as described previously.

Advantageously, this process may be directed toward affording a matt makeup result and/or homogeneous skin complexion after application.

This process may also be directed toward facilitating the spreading of the composition on the skin and leading to a uniform deposit of active agents on the skin after application. These active agents may be chosen especially from cosmetic active agents; vitamins; UV-screening agents; dyestuffs, preferably chosen from pigments and nacres; fragrances; and mixtures thereof.

Preferably, a process or a use as described above may use a composition according to the invention as described previously.

Throughout the description, including the claims, the expression “comprising a” should be understood as being synonymous with “comprising at least one”, unless otherwise specified.

The expressions “between . . . and . . . ”, “of between . . . and . . . ” and “ranging from . . . to . . . ” should be understood as meaning limits included, unless otherwise specified.

The examples and figures that follow are presented as non-limiting illustrations of the invention.

### EXAMPLES

### Example 1: Preparation of a Synthetic Phyllosilicate in Aqueous Gel Form that is Suitable for Use in the Invention

An aqueous gel of synthetic phyllosilicate according to the invention is prepared according to the technology described in example 1 of patent application FR 2 977 580 from page 21, line 26 to page 22, line 29. It was thus performed up to the formation of the hydrogel without the drying and lyophilization step.

It is this aqueous gel which is used in all the examples below.

Analysis of the x-ray diffractogram was performed using the materials and method used for the x-ray diffraction analyses that are detailed in patent application FR 2 977 580.

A characteristic diffraction line at 9.77 Å is observed.

### Example 2: Influence of the Nature of the Filler on the Matt Effect and Homogenizing Effect after Application of a Cosmetic Composition of Direct Emulsion Type (i.e. Oil-in-Water)

2.1. Compositions Tested

The phyllosilicate in aqueous gel form was introduced into the aqueous phase of an oil-in-water emulsion as defined below.

The materials of phase A1 are first weighed out and then stirred using a Moritz blender without heating. Phase A2 is then added, and the mixture is then left stirring until a homogeneous mixture is obtained.

Phases A3, A4 and A5 are then added.

Phase B is melted at 68° C. and then added into the mixture A.

The mixture thus obtained is emulsified with vigorous stirring for 10 minutes.

It is finally allowed to cool to 30° C. and phases C and D are then added.

The mixture is finally tared with water.

The following compositions were thus prepared.

The percentages indicated in this table are weight percentages relative to the total weight of the corresponding composition.

| | | | | Composition | Compo- |
|---|---|---|---|---|---|
| | | | Compo- | 2 | sition |
| | | | sition | (according | 3 |
| | | Commercial | 1 | to the | (compar- |
| | Starting materials | references | (control) | invention) | ative) |
| A1 | Microbiologically clean | | qs 100 | qs 100 | qs 100 |
| | deionized water | | | | |
| | 2-Phenoxyethanol | | 0.40% | 0.40% | 0.40% |
| | Ethylenediaminetetraacetic | Dissolvine | 0.05% | 0.05% | 0.05% |
| | acid, disodium salt, 2 H₂O | NA2-S ® | | | |
| | | (Akzo Nobel) | | | |
| A2 | 1,3-Butylene glycol | | 5.00% | 5.00% | 5.00% |
| | Xanthan gum | Rhodicare | 0.20% | 0.00% | 0.00% |
| | | XC ® | | | |
| | | (Rhodia) | | | |
| A3 | Gel of synthetic | | 0.00% | 1.80% | 0.00% |
| | phyllosilicate according | | | (i.e. 0.20% | |
| | to the invention (11% | | | AM*) | |
| | AM) in water | | | | |
| A4 | Pigments | NaI pigment | 17.06% | 17.06% | 17.06% |
| | | base | | | |
| | | (Sunpuro) | | | |
| A5 | Chlorhexidine digluconate | | 0.25% | 0.25% | 0.25% |
| | in aqueous solution | | | | |
| B | Glyceryl mono/distearate/ | Tego Care | 3.00% | 3.00% | 3.00% |
| | polyethylene glycol | 180 ® | | | |
| | stearate mixture (100 EO) | (Evonik | | | |
| | | Goldschmidt) | | | |
| | Cetylstearyl alcohol | | 1.00% | 1.00% | 1.00% |
| | (50/50 C16/C18) | | | | |
| | Hydrogenated isoparaffin | Parleam ® | 10.0% | 10.0% | 10.0% |
| | (6-8 mol of isobutylene) | (NOF | | | |
| | | Corporation) | | | |
| | Polydimethylsiloxane | Element14 | 5.00% | 5.00% | 5.00% |
| | (viscosity: 10 cSt) | PDMS 10-A ® | | | |
| | | (Momentive | | | |
| | | Performance | | | |
| | | Materials) | | | |
| | Mixture of crosslinked | KSG 16 ® | 7.00% | 7.00% | 7.00% |
| | polydimethylsiloxane and | (Shin-Etsu) | | | |
| | of polydimethylsiloxane | | | | |
| | (6 cSt) (24/76) | | | | |
| C | Natural talc | Luzenac ® | 0.00% | 0.00% | 0.20% |
| | | Pharma M | | | |
| D | Denatured absolute ethyl | | 5.00% | 5.00% | 5.00% |
| | alcohol | | | | |
| | TOTAL | | 100 | 100 | 100 |

*AM: Active Material

2.2. Evaluation of the Viscosity of Compositions 1 to 3

Protocol for Measuring the Viscosity:

Procedure

1. Thermostatization

A viscosity is significant only at a given temperature.

The measurement is taken with the assembly: product to be analyzed-cup-measuring body. It is this assembly which must be kept at the nominal temperature T ° C. 0.5° C. (in general 25° C.).

2. Zeroing

This operation will be performed, prior to each use, according to the procedure described in the operating instructions for the machine used, which in this case is a Rheomat 100.

3. Filling the Cup

Fill the cup carefully, taking care to avoid any destructuring of the product during its introduction into the cup.

The volume of substance to be introduced into each cup is as follows:

Cup of spindle 1: 320 mL measured;

Cup of spindle 2: up to the graduation mark, i.e. 60 ml;

Cup of spindles 3, 4 and 5: up to the overflow, i.e. 25 ml.

4. Measurement

Place on the machine (Rheomat 100) the support for the system and the measuring body selected, then adapt the measuring cup and switch on the machine.

Read the viscosity after 10 mins of rotation of the measuring body, the value given being in UD and requires the use of an abacus for conversion into poises.

Results:

| | Composition | Composition 2 | Composition |
|---|---|---|---|
| | 1 | (according to the | 3 |
| Viscosity | (control) | invention) | (comparative) |
| (25° C.) | 570 cps | 730 cps | 570 cps |

An increase in the viscosity of composition 2 comprising the aqueous gel of synthetic phyllosilicate according to the invention is observed, which is not the case for comparative composition 3 comprising natural talc.

Consequently, unlike the aqueous gel of synthetic phyllosilicate according to the invention, talc has no impact on the viscosity of a composition of direct emulsion type comprising it. On the other hand, an aqueous gel of synthetic phyllosilicate according to the invention behaves as a very good thickener for a composition of direct emulsion type.

2.3. Visual Evaluation of the Homogenizing Power after Application of Compositions 1 to 3

Compositions 1 to 3 are applied on an evaluation device in accordance with those described in patent application WO 2014/170807.

This device comprises an application surface containing different regions characterized by their surface state, which preferably reflects various grades of skin typology, such as skin of increasing age, skin exhibiting an increasing pore size, skin exhibiting an increasing number of skin defects and/or skin exhibiting wrinkles which are increasingly marked and/or increasingly numerous.

Each of the compositions 1 to 3 is applied by finger, at a rate of 2 mg/cm², to the application area intended to receive the sample to be evaluated.

The result obtained was then evaluated qualitatively in terms of homogenization after application.

The deposits obtained after application of these compositions were thus observed with the naked eye and using a scanning electron microscope (SEM) (4 or 5 kV at a magnification of ×100), thus making it possible to have an overall view.

The results obtained are illustrated in the table below.

| | | Composition 2 | |
|---|---|---|---|
| | Composition 1 | (according to the | Composition 3 |
| | (control) | invention) | (comparative) |
| Homogenization | — | ++ | — |
| after application | | | |

2.4. Conclusion

It is observed that composition 2 according to the invention, comprising an aqueous or aqueous-alcoholic gel of synthetic phyllosilicate according to the invention, is significantly more homogeneous after application than a composition comprising natural talc, like comparative composition 3.

More particularly, the control composition 1 has a porous and highly cracked deposit with marking of the relief.

Comparative composition 3 (i.e. containing natural talc) also has a porous and cracked deposit with accentuated marking of the relief.

In contrast, composition 2 according to the invention (i.e. comprising an aqueous gel of synthetic phyllosilicate according to the invention) shows a significantly less cracked deposit and the relief is greatly smoothed out.

2.5. Evaluation of the Correcting Effect on Skin Imperfections (Soft-Focus Effect) of Compositions 1 to 3

2.5.1. Operating Conditions

The optical properties of the test compositions are characterized by means of a Haze measurement (veil effect) with a commercial Hazemeter machine. The Haze corresponds to the scattered transmitted light/total transmitted light. The results are expressed as percentages. The greater the Haze value, the higher the soft-focus effect (Haze index).

The measurements are taken according to the following protocol:

a—On a transparent plastic film (Byk), spread a coat with a wet thickness of 25.4 μm of the composition whose Haze it is desired to evaluate is, using an automatic spreader.

b—Leave to dry for 1 hour at room temperature.

c—Measure the Haze index using a Byk Gardner brand Hazegard machine.

2.5.2. Results

2.6. Evaluation of the Matt Effect of Compositions 1 to 3

Protocol for Measuring the Mattness of a Composition

The gloss of a deposit resulting from the application of a composition may be commonly measured according to various methods, such as the method using a Byk Micro TRI gloss 20°/60°/85° glossmeter.

Principle of the Measurement Using this Glossmeter

The machine illuminates the sample to be analyzed at a certain incidence and measures the intensity of the specular reflection.

The intensity of the reflected light depends on the material and on the angle of illumination. For non-ferrous materials (paint, plastic), the intensity of reflected light increases with the angle of illumination. The rest of the incident light penetrates the material and, depending on the shade of the color, is either partly absorbed or scattered.

The reflectometer measurement results are not based on the amount of incident light but on a polished black glass standard of defined refractive index.

The measurement is normalized relative to an internal standard and brought to a value out of 100: for this calibration standard, the measurement value is set at 100 gloss units (calibration).

The closer the measured value is to 100, the more glossy the sample. The measurement unit is the Gloss Unit (GU).

The angle of illumination used has a strong influence on the reflectometer value. In order to be able to readily differentiate very glossy and matt surfaces, the standardization has defined three geometries or three measurement domains.

Test Protocol

a—Spread a coat with a wet thickness of 30 μm of the composition whose mean gloss value it is desired to evaluate onto a Leneta brand contrast card of reference Form 1A Penopac, using an automatic spreader. The coat covers the white background and the black background of the card.

b—Leave to dry for 24 hours at 37° C.

c—Measure the gloss at 20°, 60° and 85° on the matt white absorbent background (3 measurements) using a Byk Gardner brand glossmeter of reference microTri-Gloss.

The measured values in GU obtained for the various test compositions should then be compared. The lower the value measured, the more matt the deposit.

Results

Composition 2 according to the invention (i.e. comprising an aqueous gel of synthetic phyllosilicate according to the invention) has a measured GU result that is significantly lower than that obtained for the other compositions, and is consequently the composition leading to the most matt deposit.

2.7. Conclusions

An aqueous gel of synthetic phyllosilicate according to the invention can advantageously give a cosmetic composition of direct emulsion type comprising it a higher viscosity, better homogenization properties after application, and also a matt effect, especially compared with a composition comprising natural talc as filler.

In addition, the presence of a synthetic phyllosilicate makes it possible to improve the correcting effect on skin imperfections of a composition of direct emulsion type comprising it.

### Example 3: Influence of the Nature of the Filler on the Matt Effect and Homogenizing Effect after Application of a Cosmetic Composition of Water-in-Oil Inverse Emulsion Type

3.1. Compositions Tested

The materials of phase A are first weighed out and then stirred using a Moritz blender at 68° C. The pigments are then ground in the oil of phase B and then added to phase A.

Phase C is then added and the mixture thus obtained is stirred until homogeneous.

The materials of phase D are then weighed out, mixed and homogenized by stirring using a magnetic bar. Phase D is then introduced into phase A, followed by emulsifying the mixture with vigorous stirring for 10 minutes.

The mixture is finally left to cool to 30° C. and phase E is then introduced.

Taring with water is then performed.

The following compositions were then prepared.

The percentages indicated in this table are weight percentages relative to the total weight of the corresponding composition.

| | | | Compo- | Compo- |
|---|---|---|---|---|
| | | Compo- | sition 5 | sition |
| | | sition | (according | 6 |
| | Starting materials | 4 | to the | (compar- |
| | (commercial references) | (control) | invention) | ative) |
| A | PEG-10 Dimethicone | 2.00% | 2.00% | 2.00% |
| | (KSG-6017 ® from | | | |
| | Shin-Etsu) | | | |
| | Cetyl PEG/PPG-10/1 | 1.00% | 1.00% | 1.00% |
| | Dimethicone | | | |
| | (Abil EM 90 ® from | | | |
| | Evonik Goldschmidt) | | | |
| | Cyclohexasiloxane | 8.20% | 8.20% | 8.20% |
| | (KF-996 ® de | | | |
| | SHIN ETSU) | | | |
| | Isododecane | 1.00% | 1.00% | 1.00% |
| | Isohexadecane | 1.60% | 1.60% | 1.60% |
| | Ethylhexyl methoxy- | 3.00% | 3.00% | 3.00% |
| | cinnamate | | | |
| | Acrylate/polytri- | 10.0% | 10.0% | 10.0% |
| | methylsiloxy- | | | |
| | methacrylate copolymer | | | |
| | (Dow Corning FA | | | |
| | 4002 ID Silicone | | | |
| | Acrylate ® from Dow | | | |
| | Corning) | | | |
| B | NAI pigments | 12.0% | 12.0% | 12.0% |
| | (Sunpuro) | | | |
| | Cyclohexasiloxane | 7.50% | 7.50% | 7.50% |
| C | Silica | 1.00% | 1.00% | 1.00% |
| | Nylon-12 | 3.00% | 3.00% | 3.00% |
| | Perlite | 0.20% | 0.20% | 0.20% |
| D | Water | 34.80% | 16.80% | 33.33% |
| | Gel of synthetic | 0.00% | 18.00% | 0.00% |
| | phyllosilicate | | (i.e. | |
| | according to the invention | | 1.47% | |
| | (8.20% AM) in water | | AM*) | |
| | Butylene glycol | 6.00% | 6.00% | 6.00% |
| | Magnesium sulfate | 0.70% | 0.70% | 0.70% |
| | Talc naturel | 0.00% | 0.00% | 1.47% |
| | (Luzenac Pharma M) | | | |
| E | Denatured alcohol | 8% | 8% | 8% |
| | TOTAL | 100 | 100 | 100 |

*AM: Active Material

3.2. Evaluation of the Viscosity of Compositions 4 to 6

The viscosity measurement protocol is identical to that detailed in example 2 above.

Results:

| | Composition | Composition 5 | Composition |
|---|---|---|---|
| | 4 | (according to | 6 |
| | (control) | the invention) | (comparative) |
| Viscosité (25° C.) | 9 poises | 12 poises | 9 poises |

3.3. Visual Evaluation of the Homogenizing Power of Compositions 4 to 6

Compositions 4 to 6 are tested in accordance with what is indicated in example 2 above.

The results obtained are illustrated in the table below.

| | Composition | Composition 5 | Composition |
|---|---|---|---|
| | 4 | (according to | 6 |
| | (Control) | the invention) | (comparative) |
| Homogenization | −/+ | +/+ | −/+ |
| after application | | | |

3.4. Evaluation of the Matt Effect of Compositions 4 to 6

The mattness measurement protocol is identical to that described in example 2 above (see point 2.6).

Results

Composition 5 according to the invention (i.e. comprising an aqueous gel of synthetic phyllosilicate according to the invention) has a measured GU result that is lower than that obtained for the other compositions, and is consequently the composition leading to the most matt deposit.

3.5. Conclusions

A slight increase in the viscosity of composition 5 according to the invention (i.e. comprising an aqueous gel of synthetic phyllosilicate according to the invention) is observed, which is not the case for comparative composition 6 comprising natural talc.

In other words, unlike an aqueous gel of synthetic phyllosilicate according to the invention, natural talc has no impact on the viscosity of a composition of inverse emulsion type comprising it. On the other hand, an aqueous gel of synthetic phyllosilicate according to the invention behaves as a very good thickener for a composition of inverse emulsion type.

In addition, composition 5 containing the aqueous gel of synthetic phyllosilicate according to the invention is significantly more homogeneous after application than the comparative composition 6 comprising natural talc.

Finally, the presence of an aqueous gel of synthetic phyllosilicate according to the invention makes it possible to reinforce the mattness of a composition of inverse emulsion type comprising it and the persistence of this matt effect over time.

### Example 4: Influence of the Nature of the Filler on the Matt Effect and Homogenizing Effect after Application of a Cosmetic Composition of Gel-Gel Type

4.1. Compositions Tested

All the starting materials are weighed out in a beaker and then stirred vigorously using a Rayneri blender until the mixture becomes homogeneous.

Compositions 7 and 8 below were then prepared.

The percentages indicated in this table are weight percentages relative to the total weight of the corresponding composition.

| | Composition | Composition 8 | Composition |
|---|---|---|---|
| Starting materials | 7 | (according to | 9 |
| (commercial references) | (control) | the invention) | (comparative) |
| Microbiologically clean | 51.54% | 33.54% | 49.54% |
| deionized water | | | |
| Glycerol | 5.28% | 5.28% | 5.28% |
| Sodium acrylamido-2- | 2.4% | 2.4% | 3.17% |
| methylpropanesulfonate/ | | | |
| hydroxyethyl acrylate | | | |
| copolymer in powder form | | | |
| (Sepinov EMT 10 ® from | | | |
| SEPPIC) | | | |
| 2-Phenoxyethanol | 0.78% | 0.78% | 0.78% |
| Gel of synthetic | 0.00% | 18.00% | 0.00% |
| phyllosilicate according to | | (i.e. | |
| the invention at 11.0% in | | 2.00% | |
| water | | AM*) | |
| Talc naturel | 0.00% | 0.00% | 2.00% |
| (Luzenac Pharma M) | | | |
| Polydimethylsiloxane | 8.00% | 8.00% | 8.00% |
| (viscosity: 5 cSt) | | | |
| (Xiameter PMX-200 | | | |
| Silicone Fluid 5 CS ® | | | |
| from Dow Corning) | | | |
| Mixture of | 14.0% | 14.0% | 14.0% |
| polydimethylsiloxane | | | |
| crosslinked with | | | |
| hexadiene/polydimethyl- | | | |
| siloxane 5 cSt | | | |
| (Dow Corning 9041 | | | |
| Silicone Elastomer Blend ® | | | |
| from Dow Corning) | | | |
| Pigments NAI | 18.0% | 18.0% | 18.0% |
| (SUNPURO) | | | |
| TOTAL | 100 | 100 | 100 |

*AM: Active Material

4.2. Evaluation De La Viscositè Des Compositions 7 á 9

The viscosity measurement protocol is identical to that detailed in example 2 above.

Results:

| | Composition | Composition 8 | |
|---|---|---|---|
| | 7 | (according to the | Composition 9 |
| | (control) | invention) | (comparative) |
| Viscosity (25° C.) | 64 poises | 123 poises | 64 poises |

4.3. Evaluation of the Matt Effect of Compositions 7 to 9

The mattness measurement protocol is identical to that described in example 2 above.

Results

Composition 8 according to the invention (i.e. comprising an aqueous gel of synthetic phyllosilicate according to the invention) has a measured GU result that is lower than that obtained for the other compositions, and is consequently the composition leading to the most matt deposit.

4.4. Conclusions

A significant increase in the viscosity of composition 8 according to the invention (i.e. comprising an aqueous gel of synthetic phyllosilicate according to the invention) is observed, which is not the case for comparative composition 9 comprising natural talc.

In other words, unlike an aqueous gel of synthetic phyllosilicate according to the invention, talc has no impact on the viscosity of a composition of gel-gel type comprising it. On the other hand, an aqueous gel of synthetic phyllosilicate according to the invention behaves as a very good thickener for a composition of gel-gel type.

In addition, the presence of an aqueous gel of synthetic phyllosilicate according to the invention makes it possible to reinforce the mattness of a composition of gel-gel type comprising it and the persistence of this matt effect over time.

### Example 5: Influence of the Phyllosilicate Gel as a Gelling and Matt-Effect Agent for a Cosmetic Care Composition

The percentages indicated in this table are weight percentages relative to the total weight of the corresponding composition.

5.1. Compositions Tested

| | | | Composition | Compo- |
|---|---|---|---|---|
| | | Compo- | 11 | sition |
| | | sition | (according | 12 |
| Cosmetic | | 10 | to the | (compar- |
| type | INCI names | (control) | invention) | ative) |
| FATTY | Behenyl Alcohol (and) | 3.00% | 3.00% | 3.00% |
| PHASE | Glyceryl Stearate (and) | | | |
| | Disodium Ethylene Dicocamide | | | |
| | PEG-15 Disulfate (and) | | | |
| | Glyceryl Stearate Citrate | | | |
| | (Ceralution H ® from Sasol) | | | |
| | Isostearyl Neopentanoate | 2.50% | 2.50% | 2.50% |
| | (Dub VCI 18 ® from | | | |
| | Stéarineries Dubois) | | | |
| | Isohexadecane | 8.00% | 8.00% | 8.00% |
| | (Isohexadecane ® from Ineos) | | | |
| | Hexyldecyl Laurate (and) | 4.00% | 4.00% | 4.00% |
| | Hexyldecanol | | | |
| | (Cetiol PGL | | | |
| | Cognis ® (BASF)) | | | |
| | C₁₀-C₃₀ Polyalkylacrylate | 0.50% | 0.50% | 0.50% |
| | (Intelimer IPA 13-1 NG | | | |
| | Polymer ® from Air Products | | | |
| | & Chemicals) | | | |
| AQUEOUS | Synthetic phyllosilicate gel in | 0.00% | 36% SM | 0.00% |
| PHASE | accordance with the invention | | (i.e. 3.7% | |
| | (10.3% AM in water) | | AM) | |
| | Water | qs 100 | qs 100 | qs 100 |
| | Glycerol | 7.00% | 7.00% | 7.00% |
| | Preserving agent | qs | qs | qs |
| | Ammonium | 0.00% | 0.00% | 0.50% |
| | Polyacryloyldimethyl Taurate | | | |
| | (Hostacerin AMPS ® | | | |
| | Clariant) | | | |
| | Xanthan Gum | 0.20% | 0.20% | 0.20% |
| | (Rhodicare XC ® | | | |
| | Rhodia (Solvay)) | | | |

5.2. Evaluation De La Viscositè Des Compositions 10 á 12

The viscosity measurement protocol is identical to that detailed in example 2 above.

Results:

| | | Composition 11 | |
|---|---|---|---|
| | Composition 10 | (according to the | Composition 12 |
| | (control) | invention) | (comparative) |
| Viscosité (25° C.) | <1 poise | 12.2 poises | 11.5 poises |

5.3. Evaluation De l'Effet Matifiant Des Compositions 10 á 12

Le protocole de mesure de la matité est identique à celui décrit en exemple 2 ci-dessus.

Results

| | Composition | Composition 11 | Composition |
|---|---|---|---|
| | 10 | (according to the | 12 |
| | (control) | invention) | (comparative) |
| Gloss at 60° | ++++ | + | ++++++ |

Composition 11 according to the invention (i.e. comprising an aqueous gel of synthetic phyllosilicate according to the invention) has a measured GU result that is very significantly lower than that obtained for the other compositions, and is consequently the composition leading to the most matt deposit.

5.4. Conclusions

A significant increase in the viscosity of composition 11 according to the invention (i.e. comprising an aqueous gel of synthetic phyllosilicate according to the invention) is observed, which is not the case for comparative composition 12 comprising, instead of the gel according to the invention, a gelling agent not in accordance with the invention.

In addition, the presence of an aqueous gel of synthetic phyllosilicate according to the invention makes it possible to very significantly reinforce the mattness and the persistence of this matt effect over time of a composition comprising it, when compared with a composition free of such a gel.

### Example 6: Influence of the Nature of the Filler on the Spreading Properties of an Aqueous Cosmetic Composition

Les pourcentages indiqués dans ce tableau sont des pourcentages en poids du poids total de la composition correspondante.

6.1. Compositions Tested

| | | | Composition 15 |
|---|---|---|---|
| | Compo- | Compo- | (aqueous |
| | sition 13 | sition | solution |
| Commercial | (according | 14 | of sodium |
| reference | to the | (compar- | hyaluronate) |
| (supplier) | invention) | ative) | (comparative) |
| Water | qs 100 | qs 100 | qs 100 |
| Sodium hyaluronate | 1% | 1% | 1% |
| Synthetic | 18.18% | 0% | 0% |
| phyllosilicate gel | (i.e. 2% AM) | | |
| according to the | | | |
| invention | | | |
| (11% AM) | | | |
| Natural talc | 0% | 2% | 0% |
| (Luzenac ® | | | |
| Pharma M) | | | |
| Total | 100 | 100 | 100 |

6.2. Example of the Invention Compared with Counter-Example 14:

Natural talc does not disperse in the aqueous solution of composition 14 and remains at the surface. The synthetic phyllosilicate gel according to the invention, on the other hand, makes it possible to obtain a macroscopically homogeneous gel.

6.3. Homogeneity of the Deposit after Application

The reference nacre Nacre Colorona Karat Gold MP-24 is used as tracer to visually check the homogeneity of the deposit after application.

Thus, 2 g of this nacre are added to 100 g of composition 13 according to the invention and to comparative composition 15.

It is seen that the deposit obtained after application of composition 13 according to the invention is visually more homogeneous on the skin when compared with the deposit obtained with comparative composition 15 not comprising the synthetic phyllosilicate gel according to the invention.

6.4. Mattness

The mattness measurement protocol is identical to that described in example 2 above.

Composition 13 according to the invention (i.e. comprising an aqueous gel of synthetic phyllosilicate according to the invention) has a measured GU result that is very significantly lower than that obtained for the other compositions, and is consequently the composition leading to the most matt deposit.

1-24. (canceled)

25. A method for preparing a cosmetic composition using an aqueous or aqueous-alcoholic gel of synthetic phyllosilicate of formula Mg₃Si₄O₁₀(OH)₂.

26. The method as claimed in claim 25, in which the aqueous or aqueous-alcoholic gel of synthetic phyllosilicate is used as viscosity-enhancing agent.

27. The method as claimed in claim 25, in which the aqueous or aqueous-alcoholic gel of synthetic phyllosilicate is used as matt-effect agent.

28. The method as claimed in claim 25, in which the aqueous or aqueous-alcoholic gel of synthetic phyllosilicate is used as application-homogenizing agent.

29. The method as claimed in claim 25, in which the synthetic phyllosilicate of formula Mg₃Si₄O₁₀(OH)₂ has an x-ray diffraction line of greater than 9.4 Å and less than or equal to 9.8 Å.

30. A composition, comprising at least one aqueous or aqueous-alcoholic gel of synthetic phyllosilicate of formula Mg₃Si₄O₁₀(OH)₂ and at least one additional ingredient, different from said synthetic phyllosilicate gel, chosen from silicone fatty substances such as silicone oils, gums and waxes; non-silicone fatty substances such as oils, pasty substances and waxes of plant, mineral, animal and/or synthetic origin; fatty acids containing from 8 to 32 carbon atoms; synthetic esters and ethers; linear or branched hydrocarbons of mineral or synthetic origin; fatty alcohols containing from 8 to 26 carbon atoms; C₂-C₆ alcohols; glycols; surfactants; aqueous or oily gelling agents; cosmetic active agents; fragrances; fillers; dyestuffs; vitamins; preserving agents; film-forming polymers; and mixtures thereof.

31. The composition as claimed in claim 30, in which the synthetic phyllosilicate of formula Mg₃Si₄O₁₀(OH)₂ has an x-ray diffraction line of greater than 9.4 Å and less than or equal to 9.8 Å.

32. The composition as claimed in claim 30, said composition having an infrared absorption band at 7200 cm⁻¹, corresponding to the stretching vibration attributed to the silanol groups Si—OH at the edge of the phyllosilicate leaflets.

33. The composition as claimed in claim 30, said composition having no absorption band at 7156 cm⁻¹.

34. The composition as claimed in claim 30, wherein the aqueous or aqueous-alcoholic gel of synthetic phyllosilicate constitutes the aqueous phase.

35. The composition as claimed in claim 30, wherein the aqueous or aqueous-alcoholic gel of synthetic phyllosilicate is present in the composition in a content ranging from 0.01% to 40% by weight of active material relative to the total weight of the composition.

36. The composition as claimed in claim 30, wherein the aqueous or aqueous-alcoholic gel of synthetic phyllosilicate is present in the composition in a content ranging from 0.05% to 30% by weight of active material, relative to the total weight of the composition.

37. The composition as claimed in claim 30, wherein the aqueous or aqueous-alcoholic gel of synthetic phyllosilicate is present in the composition in a content ranging from 0.1% to 20% by weight of active material, relative to the total weight of the composition.

38. The composition as claimed in claim 30, wherein the aqueous or aqueous-alcoholic gel of synthetic phyllosilicate is present in the composition in a content ranging from 0.2% to 10% by weight of active material, relative to the total weight of the composition.

39. The composition as claimed in claim 30, said additional ingredient being at least one constituent compound of an aqueous phase.

40. The composition as claimed in claim 39, said additional ingredient being chosen from lower monoalcohols containing from 1 to 5 carbon atoms and polyols.

41. The composition as claimed in claim 30, said additional ingredient being at least one constituent compound of a fatty phase.

42. The composition as claimed in claim 41, said additional ingredient being chosen from a polar or apolar, volatile or non-volatile hydrocarbon based or silicone oil, a wax, a pasty compound, and a mixture thereof.

43. The composition as claimed in claim 42, said oil being at least one volatile or nonvolatile silicone oil.

44. The composition as claimed in claim 42, said hydrocarbon-based oil being chosen from volatile C₈-C₁₆ hydrocarbon-based oils, synthetic ethers containing from 10 to 40 carbon atoms, synthetic esters, polyol esters and pentaerythritol esters, fatty alcohols that are liquid at room temperature, bearing a branched and/or unsaturated carbon chain containing from 8 to 26 carbon atoms, C₁₂-C₂₂ higher fatty acids; and mixtures thereof.

45. The composition as claimed in claim 30, said additional ingredient being at least one compound chosen from nonionic, anionic or amphoteric surfactants; fillers; gelling agents; and mixtures thereof.

46. The composition as claimed in claim 30, said gelling agent being:
a hydrophilic gelling agent chosen from fumed silicas, synthetic polymeric gelling agents, polymeric gelling agents that are natural or of natural origin, acrylic or methacrylic acid homopolymers or copolymers, salts thereof or esters thereof, polyacrylic acid/alkyl acrylate copolymers, crosslinked or non-crosslinked polyoxyethylenated AMPS/alkyl methacrylate copolymers, anionic, cationic, amphoteric or nonionic chitin or chitosan polymers, cellulose polymers, other than alkylcellulose, chosen from hydroxyethylcellulose, hydroxypropylcellulose, hydroxymethylcellulose, ethylhydroxyethylcellulose, carboxymethylcellulose, and also quaternized cellulose derivatives, vinyl polymers and anionic, cationic, nonionic or amphoteric associative polymers, modified or unmodified polymers of natural origin, alginates and carrageenans, glycoaminoglycans, deoxyribonucleic acid, mucopolysaccharides and chondroitin sulfates, and mixtures thereof;
a lipophilic gelling agent chosen from particulate gelling agents, organopolysiloxane elastomers, semicrystalline polymers, hydrocarbon-based block copolymers, dextrin esters and polymers bearing hydrogen bonding, and mixtures thereof;
and mixtures thereof.

47. The composition as claimed in claim 30, said additional ingredient being at least one compound chosen from cosmetic active agents; vitamins; UV-screening agents; dyestuffs; fragrances; and mixtures thereof.

48. The composition as claimed in claim 30, wherein said composition is in the form of a composition for caring for and/or making up the skin and/or the nails, of the body or of the face, and is preferably a foundation, a face powder or an eyeshadow.

49. The composition as claimed in claim 30, wherein said composition is a foundation, a face powder or an eyeshadow.

50. The composition as claimed in claim 30, wherein said composition is intended for caring for and/or making up the lips.

51. The composition as claimed in claim 30, wherein said composition is a stick, a varnish or a gloss.

52. A process for making up and/or caring for the skin and/or the nails, comprising at least one step of applying to said skin and/or said nails a composition comprising, in a physiologically acceptable medium, at least one aqueous or aqueous-alcoholic gel of synthetic phyllosilicate of formula Mg₃Si₄O₁₀(OH)₂.

53. The process as claimed in claim 52, in which said composition comprises at least one aqueous or aqueous-alcoholic gel of synthetic phyllosilicate of formula Mg₃Si₄O₁₀(OH)₂ and at least one additional ingredient, different from said synthetic phyllosilicate gel, chosen from silicone fatty substances such as silicone oils, gums and waxes; non-silicone fatty substances such as oils, pasty substances and waxes of plant, mineral, animal and/or synthetic origin; fatty acids containing from 8 to 32 carbon atoms; synthetic esters and ethers; linear or branched hydrocarbons of mineral or synthetic origin; fatty alcohols containing from 8 to 26 carbon atoms; C₂-C₆ alcohols; glycols; surfactants; aqueous or oily gelling agents; cosmetic active agents; fragrances; fillers; dyestuffs; vitamins; preserving agents; film-forming polymers; and mixtures thereof.

54. The process as claimed in claim 52, said process being directed toward affording a matt and/or homogeneous makeup result for the skin complexion after application.

55. The process as claimed in claim 52, said process being directed toward facilitating the spreading of the composition on the skin and toward leading to a homogeneous deposit of active agents on the skin after application.

56. The process as claimed in claim 55, wherein said active agents are chosen from cosmetic active agents; vitamins; UV-screening agents; dyestuffs; fragrances; and mixtures thereof.
